SEQUENCE LISTING (7 sequences)

SEQ ID NO: 1 - DNA, 2205 bp, Mus sp.
gaccggtaacaagtggccgggagcgaacttttgcaaatctcttctgcgccttaaggctgccaccgagactgtaaagaaaagggagaagaggaacctatactcataccagttcgcacaggcggctgaagttgggcgagcgctagccgcggctgcctagcgtccccctccccctcacagcggaggaggggacagttgtcggaggccgggcggcagagcccgatcgcgggcttccaccgagaattccgtgacgactggtcagcaccgccggagagccgctgttgctgggactggtctgcgggctccaaggaaccgctgctccccgagagcgctccgtgagtgaccgcgacttttcaaagctcggcatcgcgcgggagcctaccaacgtgagtgctagcggagtcttaaccctgcgctccctggagcgaactggggaggagggctcagggggaagcactgccgtctggagcgcacgctcctaaacaaactttgttacagaagcggggacgcgcgggtatccccccgcttcccggcgcgctgttgcggccccgaaacttctgcgcacagcccaggctaaccccgcgtgaagtgacggaccgttctatgactgcaaagatggaaacgaccttctacgacgatgccctcaacgcctcgttcctccagtccgagagcggtgcctacggctacagtaaccctaagatcctaaaacagagcatgaccttgaacctggccgacccggtgggcagtctgaagccgcacctccgcgccaagaactcggaccttctcacgtcgcccgacgtcgggctgctcaagctggcgtcgccggagctggagcgcctgatcatccagtccagcaatgggcacatcaccactacaccgacccccacccagttcttgtgccccaagaacgtgaccgacgagcaggagggcttcgccgagggcttcgtgcgcgccctggctgaactgcatagccagaacacgcttcccagtgtcacctccgcggcacagccggtcagcggggcgggcatggtggctcccgcggtggcctcagtagcaggcgctggcggcggtggtggctacagcgccagcctgcacagtgagcctccggtctacgccaacctcagcaacttcaacccgggtgcgctgagcagcggcggtggggcgccctcctatggcgcggccgggctggcctttccctcgcagccgcagcagcagcagcagccgcctcagccgccgcaccacttgccccaacagatcccggtgcagcacccgcggctgcaagccctgaaggaagagccgcagaccgtgccggagatgccgggagagacgccgcccctgtcccctatcgacatggagtctcaggagcggatcaaggcagagaggaagcgcatgaggaaccgcattgccgcctccaagtgccggaaaaggaagctggagcggatcgctcggctagaggaaaaagtgaaaaccttgaaagcgcaaaactccgagctggcatccacggccaacatgctcagggaacaggtggcacagcttaagcagaaagtcatgaaccacgttaacagtgggtgccaactcatgctaacgcagcagttgcaaacgttttgagaacagactgtcagggctgaggggcaatggaagaaaaaaaataacagagacaaacttgagaacttgactggttgcgacagagaaaaaaaaagtgtccgagtactgaagccaagggtacacaagatggactgggttgcgacctgacggcgcccccagtgtgctggagtgggaaggacgtggcgcgcctggctttggcgtggagccagagagcagcggcctattggccggcagactttgcggacgggctgtgcccgcgcgcgaccagaacgatggacttttcgttaacattgaccaagaactgcatggacctaacattcgatctcattcagtattaaaggggggtgggaggggttacaaactgcaatagagactgtagattgcttctgtagtgctccttaacacaaagcagggagggctgggaaggggggggaggcttgtaagtgccaggctagactgcagatgaactcccctggcctgcctctctcaactgtgtatgtacatatatatttttttttaatttgatgaaagctgattactgtcaataaacagcttcctgcctttgtaagttattccatgtttgtttgtttgggtgtcctgccc

SEQ ID NO: 2 - DNA, 22 bp, Mus sp.
  misc_feature (1)..(22): Forward Primer
gagtgctagcggagtcttaacc

SEQ ID NO: 3 - DNA, 18 bp, Mus sp.
  misc_feature (1)..(18): Reverse Primer
ctccagacggcagtgctt

SEQ ID NO: 4 - DNA, 18 bp, Mus sp.
  misc_feature (1)..(18): Probe
aagcactgccgtctggag

SEQ ID NO: 5 - DNA, 620 bp, Mus sp.
cctttggtctctggagtgctggtgtatgctgaaagtgatcaaaagagtttctcttccccccatgtccccatccttcagaagtgaaggggagcagccctgggccctgctctggcggatgctgtggtgggaaggaagtggctaaagggttgctgggcctcttctttgagaccttagtggtggtgtctttcatgtagcacatgctggaggctgggagccagtgagtctttcctgtgaggggtctttcaggagctgaattgctcagcagacctgaatgaagaaatggctacatgtaagccaggtccaccttgctccaaaagaaagtgattctctctctctctctctctctctctcacacacacacacacacacacacacacacacacaactttctttatagttttattgtggcagcctctcagagggtcagttgtttgtttaagatacactcaattaatgatatgcttgcttcatattgtgtctttcagaggagaaaggtaactgagaccccactgctcacccgctgtattgttaagccagtgaataggaacccaaaaggacagagaagacacccaacttaactcaccatgcacgagtctctgagcctcaggaacttgaattta

SEQ ID NO: 6 - DNA, 20 bp, Mus sp.
  misc_feature (1)..(20): Forward Primer
ccaccttgctccaaaagaaa

SEQ ID NO: 7 - DNA, 20 bp, Mus sp.
  misc_feature (1)..(20): Reverse Primer
tattgtggcagcctctcaga

(19) United States
(12) Patent Application Publication
(10) Pub. No.: US 2005/0272085 A1
(43) Pub. Date: Dec. 8, 2005

(54) Methods for forensic and congenic screening

(76) Inventor: Timothy A. Hodge, Eads, TN (US)

Correspondence Address: BUTLER, SNOW, O'MARA, STEVENS & CANNADA PLLC, 6075 POPLAR AVENUE, SUITE 500, MEMPHIS, TN, 38119, US

(21) Appl. No.: 11/170,477

(22) Filed: Jun. 29, 2005

Related U.S. Application Data
(63) Continuation-in-part of application No. 11/074,995, filed on Mar. 8, 2005.
(63) Continuation-in-part of application No. 09/945,952, filed on Sep. 4, 2001.
(60) Provisional application No. 60/230,371, filed on Sep. 6, 2000.

(51) Int. Cl. C12Q 1/68; G06F 19/00; G01N 33/48; G01N 33/50

(52) U.S. Cl. 435/6; 702/20

(57) ABSTRACT

The present invention provides a method to rapidly provide genotype screening of a plurality of samples for a microsatellite loci. The screening results have forensic and congenic applications.

### CROSS-REFERENCE TO RELATED APPLICATIONS

This application claims priority under 35 U.S.C. §120 as a CONTINUATION-IN-PART APPLICATION of a co-pending application entitled “System, Method and Apparatus for Transgenic and Targeted Mutagenesis Screening” which was filed on Sep. 4, 2001, and was assigned U.S. App. Ser. No. 09/945,952 (the “'952 Application”), U.S. patent application Ser. No. 11/074,995 filed Mar. 8, 2005, and U.S. patent application Ser. No. ______ filed Jun. 24, 2005, entitled “Methods for Genotype Screening” the entire disclosures of which are incorporated herein by reference for all that it teaches. This application and the '952 Application also claim priority under 35 U.S.C. §119(e), based on U.S. Provisional Application Ser. No. 60/230,371, filed Sep. 6, 2000, the entire disclosure of which is incorporated herein by reference for all that it teaches.

### FIELD OF THE INVENTION

This invention relates to methods for forensic and congenic screening. More specifically, this invention relates to various methods to detect or screen for at least one designated genetic sequences in a plurality of biological samples. In the preferred embodiment the biological sample is disposed on an adsorbent carrier or a tissue sample.

### BACKGROUND OF THE INVENTION

Microsatellite loci, generally known in forensic applications as Short Tandem Repeat (STR) loci, are widely used for forensic identification and relatedness testing, and are a predominant genetic marker in this area of application. In forensic identification cases, the goal is typically to link a suspect with a sample of blood, semen or hair taken from a crime or victim. Alternatively, the goal may be to link a sample found on a suspect's clothing with a victim. Relatedness testing in criminal work may involve investigating paternity in order to establish rape or incest. Another application involves linking DNA samples with relatives of a missing person. Because the lengths of microsatellites may vary from one person to the next, scientists have begun to use them to identify criminals and to determine paternity, a procedure known as DNA profiling or “fingerprinting”. The features that have made use of microsatellites attractive are due to their relative ease of use, accuracy of typing and high levels of polymorphism. The ability to employ PCR to amplify small samples is particularly valuable in this setting, since in criminal casework only minute samples of DNA may be available. Similarly, because microsatellites change in length early in the development of some cancers, they are useful markers for early cancer detection. Because they are polymorphic they are useful in linkage studies which attempt to locate genes responsible for various genetic disorders. Additionally, by looking at the variation of microsatellites in populations, inferences can be made about population structures and differences, genetic drift, genetic bottlenecks and even the date of a last common ancestor. Microsatellites can be used to detect sudden changes in population, effects of population fragmentation and interaction of different populations. Microsatellites are useful in identification of new and incipient populations.

Congenic strains are mouse strains that carry a mutant or polymorphic allele from one strain, on a different strain. The mice are created by mating the donor strain, which are the mice with a mutation or foreign genetic sequence, to a specific recipient inbred strain. After 10 generations of backcrossing to recipient inbred strain the fully congenic strain is expected to be identical at all loci except for the mutation.

The mouse genome has been extensively mapped using microsatellite markers in at least 54 inbred strains. Microsatellites are repeat elements that occur in the mouse genome in non-coding regions. Inbred strains frequently differ from one another in the number of these repeat units at specific sites in the genome. These repeat elements are the designated genetic sequence. Designing PCR primers that flank the designated genetic sequence allows for discrimination of the number of repeat units using fragment analysis.

A genotyping screening strategy utilizing a panel of microsatellites that are polymorphic between the donor and recipient strain, allows the inbred strains to be distinguished from one another. These polymorphic loci span the entire genome with the exception of the sex chromosomes. There are more than 6,000 microsatellite markers that are commonly used in the mapping of mice.

Alternatively, a genotype screening strategy may be employed that utilizes single nucleotide polymorphisms (SNP) between the donor and recipient inbred strain. There are greater than 3 million SNP markers identified in humans. With additional inbred mouse strains being sequenced it will give rise to a tremendous amount of SNPs that will be used for marker assisted breeding. This population of SNPs distributed throughout the genome will provide greater resolution of genetic mapping of genomes.

Marker Assisted Breeding or Speed Congenics utilizes genotyping to setup specific breedings between donors and recipient inbred mice. The progeny that have the highest percentage of the recipient genome, while still maintaining the mutation, are selected for the next round of backcrossing. Through specific genotype profiling of the breeders, it is possible to reach 99% recipient strain genomic identities after five generations.

Genotype screening is currently done manually. The present manual system is time-consuming and can provide variable results depending on the laboratory and even depending on skill of laboratory workers. Manual nucleic acid isolations, PCR amplification, amplicon quantification and capillary electrophoresis of up to 30 samples can take most laboratories 3 to 7 days. A need exists in the industry to provide a system and method for more accurate, faster and high volume genotype screening.

### SUMMARY OF THE INVENTION

The present invention provides a unique solution to the above-described problems by providing a method for rapid genotype screening. In particular, this invention provides a method to rapidly report screening results to a remote user from a screening laboratory for a plurality of biological samples either tissue or disposed on an adsorbent carrier. Efficient screening of a plurality of biological samples can be achieved by placing the sample to be screened in a well of a microwell container. More specifically, this invention discloses a method to screen plurality of samples for microsatellite loci. The method includes, the steps of: acquiring the identity of at least one microsatellite loci for a said plurality of samples; obtaining means to determine the presence of said microsatellite loci; and receiving at a screening laboratory from a remote user a plurality of samples disposed in a designated well of a microwell container, and screening said plurality of samples for at least one microsatellite loci.

### BRIEF DESCRIPTION OF THE DRAWINGS

A more complete understanding of the invention and its advantages will be apparent from the following Description of the Preferred Embodiment(s) taken in conjunction with the accompanying drawings, wherein:

FIG. 1 is an illustrative overview of the remote automated testing procedures of the present invention.

FIG. 2 is a block diagram of one embodiment of the system.

FIG. 3 is a block diagram of the ordering procedure.

FIG. 4 is a block diagram of account registration.

FIGS. 5-6 illustrate the survey of work and sample identification sections.

FIG. 7A is a block diagram of the laboratory process system.

FIG. 7B is a block diagram of the laboratory process system.

FIG. 7C is a block diagram of the laboratory process system.

FIG. 7D is a block diagram of the laboratory process system.

FIG. 8 is a block diagram of standard laboratory stations.

FIG. 9 is a screen display illustrating a document on the transgenic screening laboratory 20's web site relating to an outcome file.

FIG. 10 is a graphical representation of the results.

FIG. 11 is a graphical representation of signal magnitude.

FIG. 12 is a graphical representation of signal magnitude.

FIG. 13 is a graphical representation of signal magnitude.

FIGS. 14 and 15 illustrate a preferred device for performing the functions of a Lysing Station and an Automated Accessioning Station as described herein, including an oven (FIG. 15) for incubating the samples.

FIG. 16 illustrates a preferred device for performing the functions of an Isolation/Purification Station as described herein.

FIG. 17 illustrates a preferred device for drying samples.

FIG. 18 illustrates a preferred device for performing the functions of a Screening Station as described herein.

FIG. 19 illustrates a preferred device for performing the functions of a Detection Station as described herein.

FIG. 20A shows a schematic diagram of two swab holders.

FIG. 20B shows a cross-sectional view of an swab holder.

FIG. 21 shows a schematic diagram of a kit.

FIG. 22 shows a schematic diagram of an electrophoresis device.

FIGS. 23-33 show a representative screening result for human data.

### DETAILED DESCRIPTION OF THE PREFERRED EMBODIMENTS

The present invention provides a method for high volume genotype screening. This invention provides a method for rapid identification of an organism, whose genome possesses specific genetic sequences that exist endogenously or has been modified, mutated or genetically engineered. All patents, patent applications and articles discussed or referred to in this specification are hereby incorporated by reference.

1. Definitions

The following terms and acronyms are used throughout the detailed description.

complementary—chemical affinity between nitrogenous bases as a result of hydrogen bonding. Responsible for the base pairing between nucleic acid strands. Klug, W. S. and Cummings, M. R. (1997) Concepts of Genetics, fifth ed., Prentice-Hall, Upper Saddle River, N.J.

congenics—Strains generated by repeated backcrossing that differ from one another only with respect to a small chromosomal segment. Many congenic mouse strains differ only in a segment containing the major histocompatibility complex D.

copy number—the number of transgenes that have randomly integrated into the genome.

Cjun—(housekeeping or reference sequence)

(SEQ ID NO. 1)

GACCGGTAACAAGTGGCCGGGAGCGAACTTTTGCAAATCTCTTCTGCGCC
TTAAGGCTGCCACCGAGACTGTAAAGAAAAGGGAGAAGAGGAACCTATAC
TCATACCAGTTCGCACAGGCGGCTGAAGTTGGGCGAGCGCTAGCCGCGGC
TGCCTAGCGTCCCCCTCCCCCTCACAGCGGAGGAGGGGACAGTTGTCGGA
GGCCGGGCGGCAGAGCCCGATCGCGGGCTTCCACCGAGAATTCCGTGACG
ACTGGTCAGCACCGCCGGAGAGCCGCTGTTGCTGGGACTGGTCTGCGGGC
TCCAAGGAACCGCTGCTCCCCGAGAGCGCTCCGTGAGTGACCGCGACTTT
TCAAAGCTCGGCATCGCGCGGGAGCCTACCAACGTGAGTGCTAGCGGAGT
CTTAACCCTGCGCTCCCTGGAGCGAACTGGGGAGGAGGGCTCAGGGGGAA
GCACTGCCGTCTGGAGCGCACGCTCCTAAACAAACTTTGTTACAGAAGCG
GGGACGCGCGGGTATCCCCCCGCTTCCCGGCGCGCTGTTGCGGCCCCGAA
ACTTCTGCGCACAGCCCAGGCTAACCCCGCGTGAAGTGACGGACCGTTCT
ATGACTGCAAAGATGGAAACGACCTTCTACGACGATGCCCTCAACGCCTC
GTTCCTCCAGTCCGAGAGCGGTGCCTACGGCTACAGTAACCCTAAGATCC
TAAAACAGAGCATGACCTTGAACCTGGCCGACCCGGTGGGCAGTCTGAAG
CCGCACCTCCGCGCCAAGAACTCGGACCTTCTCACGTCGCCCGACGTCGG
GCTGCTCAAGCTGGCGTCGCCGGAGCTGGAGCGCCTGATCATCCAGTCCA
GCAATGGGCACATCACCACTACACCGACCCCCACCCAGTTCTTGTGCCCC
AAGAACGTGACCGACGAGCAGGAGGGCTTCGCCGAGGGCTTCGTGCGCGC
CCTGGCTGAACTGCATAGCCAGAACACGCTTCCCAGTGTCACCTCCGCGG
CACAGCCGGTCAGCGGGGCGGGCATGGTGGCTCCCGCGGTGGCCTCAGTA
GCAGGCGCTGGCGGCGGTGGTGGCTACAGCGCCAGCCTGCACAGTGAGCC
TCCGGTCTACGCCAACCTCAGCAACTTCAACCCGGGTGCGCTGAGCAGCG
GCGGTGGGGCGCCCTCCTATGGCGCGGCCGGGCTGGCCTTTCCCTCGCAG
CCGCAGCAGCAGCAGCAGCCGCCTCAGCCGCCGCACCACTTGCCCCAACA
GATCCCGGTGCAGCACCCGCGGCTGCAAGCCCTGAAGGAAGAGCCGCAGA
CCGTGCCGGAGATGCCGGGAGAGACGCCGCCCCTGTCCCCTATCGACATG
GAGTCTCAGGAGCGGATCAAGGCAGAGAGGAAGCGCATGAGGAACCGCAT
TGCCGCCTCCAAGTGCCGGAAAAGGAAGCTGGAGCGGATCGCTCGGCTAG
AGGAAAAAGTGAAAACCTTGAAAGCGCAAAACTCCGAGCTGGCATCCACG
GCCAACATGCTCAGGGAACAGGTGGCACAGCTTAAGCAGAAAGTCATGAA
CCACGTTAACAGTGGGTGCCAACTCATGCTAACGCAGCAGTTGCAAACGT
TTTGAGAACAGACTGTCAGGGCTGAGGGGCAATGGAAGAAAAAAAATAAC
AGAGACAAACTTGAGAACTTGACTGGTTGCGACAGAGAAAAAAAAAGTGT
CCGAGTACTGAAGCCAAGGGTACACAAGATGGACTGGGTTGCGACCTGAC
GGCGCCCCCAGTGTGCTGGAGTGGGAAGGACGTGGCGCGCCTGGCTTTGG
CGTGGAGCCAGAGAGCAGCGGCCTATTGGCCGGCAGACTTTGCGGACGGG
CTGTGCCCGCGCGCGACCAGAACGATGGACTTTTCGTTAACATTGACCAA
GAACTGCATGGACCTAACATTCGATCTCATTCAGTATTAAAGGGGGGTGG
GAGGGGTTACAAACTGCAATAGAGACTGTAGATTGCTTCTGTAGTGCTCC
TTAACACAAAGCAGGGAGGGCTGGGAAGGGGGGGGAGGCTTGTAAGTGCC
AGGCTAGACTGCAGATGAACTCCCCTGGCCTGCCTCTCTCAACTGTGTAT
GTACATATATATTTTTTTTTAATTTGATGAAAGCTGATTACTGTCAATAA
ACAGCTTCCTGCCTTTGTAAGTTATTCCATGTTTGTTTGTTTGGGTGTCC
TGCCC

(SEQ ID NO. 2)

Forward Primer: GAGTGCTAGCGGAGTCTTAACC

(SEQ ID NO. 3)

Reverse Primer: CTCCAGACGGCAGTGCTT

(SEQ ID NO. 4)

Probe: AAGCACTGCCGTCTGGAG

designated genetic sequence—includes a transgenic insert, a selectable marker, microsatellite loci, recombinant site or any gene or gene segment.

DNA (deoxyribonucleic acid)—One of the two main types of nucleic acid, consisting of a long, unbranched macromolecule formed from one, or more commonly, two, strands of linked deoxyribonucleotides, the 3″-phosphate group of each constituent deoxyribonucleotide being joined in 3′,5′-phosphodiester linkage to the 5′-hydroxyl group of the deoxyribose moiety of the next one. Oxford Dictionary of Biochemistry and Molecular Biology; p. 182.

embryonic stem cells (ES cells)—a cell of the early embryo that can replicate indefinitely and which can differentiate into other cells; stem cells serve as a continuous source of new cells.

genome—all the genetic material in the chromosomes of a particular organism; its size is generally given as its total number of base pairs.

genomic nucleic acid—The genomic nucleic acid includes both coding and noncoding regions. Therefore, the genomic nucleic acid contains exons and introns, promoter and gene regulation regions, telomeres, origins or replication and nonfimctional intergenic nucleic acid. The genomic nucleic acid is a double stranded molecule which is methylated. cDNA and PCR-amplicons differs in that the molecules are much smaller. Additionally, biochemical modification events, such as methylation, do not occur with the smaller molecules. Shena, M (2000) DNA Microarrays: A Practical Approach. Oxford University Press, New York, N.Y.

genotype—genetic constitution of an individual cell or organism that can include at least one designated gene sequence.

hemizygous—a situation within a cell or organism where only one copy of a gene, group of genes or genetic sequence is present instead of two copies in a diploid genome.

heterozygosity—the state of having two different genes (alleles) at one or more corresponding loci on homologous chromosomes.

homozygosity—The state of having the same genes (alleles) at one or more corresponding homologous chromosomes.

internet—a collection of interconnected (public and/or private) networks that are linked together by a set of standard protocols to form a global, distributed network. The World Wide Web (hereinafter web) refers to both a distributed collection of interlinked, user viewable hypertext documents (commonly referred to as web pages) that are accessible via the Internet and the user and server software components which provide user access to such documents using standard Internet protocols.

line—A line is a group of organisms bred for a genotype (i.e. at least one designated genetic sequence).

Microsatellite is a specific sequence of DNA bases or nucleotides which contain mono, di, Tri or tetra repeats. In the literature they can also be called simple sequence repeats (SSR), short tanden repeats (STR), or variable number tandem repeats (VNTR). Alleles at a specific location (locus) can differ in the number of repeats. Microsatellites are inherited in a Mendelian fashion.

mutation—a heritable change in DNA sequence resulting from mutagens. Various types of mutations including frame-shift mutations, missense mutations, and nonsense mutations.

plate controls—are wells that include the house-keeping probe without nucleic acid sample.

recombination—The process by which offspring derive a combination of genes different from that of either parent. In higher organisms, this can occur by crossing over.

recombinant DNA—A combination of DNA molecules of different origin that are joined using recombinant DNA technologies.

RNA—on of the two main types of nucleic acid, consisting of a long, unbranched macromolecule formed from ribonucleotides, the 3′-phosphate group of each constituent ribonucleotide (except the last) being joined in 3′,5′-phosphodiester linkage to the 5′-hydroxyl group on each ribose moiety renders these phosphodiester bonds susceptible to hydrolytic attack by alkali, in contrast to those of DNA. The RNA chain has polarity, with one 5′ end and on 3′ end. Two purines, adenine and guanine, and two pyrimidines, cytosine and uracil, are the major bases usually present. In addition, minor bases may occur; transfer RNA, however, contains unusual bases in relatively large amounts. The sequence of bases carries information, whereas the sugar and phosphate groups play a structural role. RNA is fundamental to protein biosynthesis in all living cells. Oxford Dictionary of Biochemistry and Molecular Biology; p. 577.

screening reference—are probes that are run on every sample submitted to screen laboratory. The probe is one that is found in every mouse, mutant or not.

strain—a group of organisms bred for a genotype (at least one designated genetic sequence).

strain controls—are biomatter samples submitted by a remote user 1. Strain controls are controls positive and negative sent to the screen laboratory as the remote user that discloses the genotype.

transgene—the foreign gene or DNA.

transgenic—this term describes an organism that has had genes from an organism or additional elements of it our sequence put into its genome through recombinant DNA techniques. These organisms are usually made by microinjection of DNA in the pronucleus of fertilized eggs, with the DNA integrating at random.

transgenic line—a transgenic mouse or organism strain in which the transgene is stably integrated into the germline and therefore inherited in Mendelian fashion by succeeding generation.

web site—a computer system that serves informational content over a network using the standard protocol of the World Wide Web. A web site corresponds to a particular Internet domain name such as TransnetYX.com.

wild type—the phenotype that is characteristic of most of the members of a species occurring naturally and contrasting with the phenotype of a mutant.

zygosity—This term reflect the genetic makeup of an individual. When identical alleles exist at a loci it is said to be homozygous; when alleles are different the alleles are said to be heterozygous.

2. Overview of the Systems Components and Operations

The present invention provides methods for genotype screening. More specifically, the present application relates to a method to rapidly screen biological samples for at least one designated genetic sequence. Various aspects of genotype screening involve: sample collection, lysing of the biological sample, isolation of purified genomic nucleic acid and nucleic acid screening. Additionally, the method operating according to the features described herein can provide screening results to a remote user 1 from the screening laboratory 20 within 24 hours of receiving the biological samples.

In order to screen for a designated genetic sequence, that sequence must first be determined or identified. Only when the designated sequence is known can a test be devised to search for its existence in the biological samples provided by the remote user 1 to the screening laboratory 20.

There are a variety of ways the designated genetic sequence can be acquired by the remote user 1 or by the screening laboratory 20. For example, if the sequence of bases that makeup the designated genetic sequence is known by the remote user 1, the sequence can be directly communicated to the screening laboratory 20 via an electronic link, such as any of the electronic communication links identified herein, and particularly the communication links extending between the remote user's computer and the screening laboratory 20.

One of the simplest methods for identifying informative microsatellites involves the use of public databases and vendor provided reagents. The screening laboratory 20 or the remote user 1 has the ability to identify which microsatellites are informative between two inbred strains by using public databases such as the one housed by the The Center for Inherited Disease Research http://www.cidr.jhmi.edu/. By selecting different strains, the database creates a panel of microsatellites that will distinguish one inbred strain from another. The fluorescently labeled PCR primers that are used to amplify the microsatellites are available from Applied Biosystems. Currently there are 314 proprietary PCR primer sets for mouse mapping offered by Applied Biosystems.

The remote user 1 can indirectly communicate the designated genetic sequence to the screening laboratory 20 by communicating a publication, journal article, a gene name, a sequence name, a line or strain name (if the designated genetic sequence is found in animals of that line or strain), or the name of a mutation having the designated genetic sequence to the screening laboratory 20. Alternatively, the remote user 1 can communicate to the screening laboratory 20 the sequence of a primer set or probe that corresponds to a target genetic sequence of the designated genetic sequence. These primer sets or probes will have previously been created or defined to indicate the presence of the designated genetic sequence.

The indirect references may provide the entire sequence. Alternatively, the screening laboratory 20 may take the information from the references or from the remote user 1 and use it to search public genetic databases such as The National Center for Biotechnology Information (NCBI), Ensembl, or The Wellcome Trust Sanger Institute database. The screening laboratory 20 can also search proprietary databases, such as the database provided by Celera Bioscience (Rockville, Md.).

If the designated genetic sequence is not known by the remote user 1 or third party and is not found in any public or private database, the screening laboratory 20 may use scientific methods. If the remote user 1 has a working genotyping assay, and they are performing PCR and separating fragments in a gel, the appropriate bands can be cut from the gel, purified and sequenced to determine the sequence of bases in that band. The company sequencing the bands can directly communicate the base sequence to the screening laboratory 20 or to the remote user 1, who in turn can communicate the base sequence to the screening laboratory 20.

Once identity of the designated genetic sequence is acquired by the screening laboratory 20 (and assuming primer set has yet to be designed), the screening laboratory 20 must then select a target genetic sequence of the designated genetic sequence for which a primer set can be constructed. In the preferred embodiment, the sequence of the primer set is determined using software such as Primer Express® (Applied Bio Systems). The target genetic sequence may be directly selected from the designated genetic sequence by the screening laboratory 20. Once selected, the base sequence corresponding to the target genetic sequence is communicated to an oligonucleotide vendor, who manufactures the primer sets and transmits them to the screening laboratory 20.

The screening laboratory 20 preferably keeps a supply of primer sets on hand so each future request by the remote user need not require special production of primer sets.

Alternatively, a special probe or primer set may be required. In that situation, the screening laboratory 20 may not select the target genetic sequence itself, but may communicate to a third party specific areas in the designated genetic sequence that are important for detection. The third party is typically an oligonucleotide vendor, who in turn will select the target genetic sequence, manufacture the primer sets, and send the probes and primer sets to the screening laboratory 20.

With respect to human genotyping, a remote user 1 can contact the screening laboratory 20 and provide information for a human mutation or suspected endogenous condition of interest. This information may include the remote user's interest in wanting to know if the sample is from a human or a mouse and if it is from a human what gender is the sample. The screening laboratory 20 can acquire primers and that can distinguish between humans and mice. This is accomplished by identifying areas of genetic sequence in the mouse genome that are not homologous with the genetic sequence in the Homo sapiens genome. With no input from the remote user 1, the screening laboratory 20 can query a database such as Ensembl that would discriminate between the sex chromosomes in humans (X and Y). This query would yield sequence data for the Y chromosome, which is the designated genetic sequence. The screening laboratory 20 can take the designated genetic sequence, or portion thereof, and build the primer set as to be informative for screening. The remote user 1's Internet web-based account will have a field populated that represents these reagents with an identifier such as the genetic line/profile identification 84. The remote user 1 will use the identifier (strain name or profile name) to indicate that these specific reagents are to be used on subsequent samples.

Similarly, if the remote user 1 requires SNP genotyping a remote user 1 can contact the screening laboratory 20 and provide a literature reference of the mutation which discloses the mutation name. A mutation name query of the Mouse Genome Informatics website, Ensembl or National Center for Biotechnology Information that provides sequence data. This sequence data is the designated genetic sequence. Knowing the endogenous nucleotide and the mutant nucleotide, the screening laboratory 20 can take the designated genetic sequence, or portion thereof, and send it to a vendor indicating specifically where to build the primers and probes as to be informative for screening. For example, if the designated genetic sequence is 500 nucleotides in length, the screening laboratory 20 may indicate to the reagent vendor to build a SNP assay targeting the 239^{th} nucleotide. The reagent vendor will then supply to the screening laboratory 20, the primers and probes to specifically discriminate between a nucleotide change at the 239^{th} position of the designated genetic sequence.

Specifically, a remote user 1 can contact the screening laboratory 20 and request that specific microsatellite assays be performed on samples. These microsatellite detection reagents may be supplied to the screening laboratory from vendors.

The remote user 1's Internet web-based account will have a field populated that represents these reagents with an identifier such as a name or number, or what is commonly referred to as the genetic line/profile identification 84. The remote user 1 will use the genetic line identification 84 to indicate that these specific reagents are to be used on subsequent samples.

The primer sets, if they are new and have not before been tested against a sample containing the designated genetic sequence, must then be tested, preferably by the screening laboratory 20. To do this, the screening laboratory 20 preferably receives both a positive and a negative strain control samples from the remote user 1 and tests them against the probes and primer sets to confirm that they can be used successfully to determine whether the designated genetic sequence can be detected. These controls include one positive and one negative control for each mutation found in the strain of interest.

If the designated genetic sequence can be detected using the primer sets, the screening laboratory 20 updates the website and the order management software to provide the remote user 1 with a web-based selection for sample testing using those tested primer sets. These selections are among those which the remote user 1 can select from the screening parameter selections identified below.

Alternatively, for example, if the remote user 1 or other third party communicates to the screening laboratory 20 that a particular primer set has already been tested and is known to work, or if the screening laboratory 20 has already designed a primer set for the designated genetic sequence (which is commonly the case for often-used strains or lines) the screening laboratory 20 can immediately add a selection to the website and does not need to test controls with the primer sets.

The strain controls are used to tell LIMS 24 a signal magnitude that is then associated with a positive or negative sample. In one case, the remote user 1 may send these controls together with the samples to be tested to the screening laboratory 20 in a single shipment. Alternatively, the controls may be sent separately from the samples to be tested.

The screening laboratory 20 tests the strain controls using the process described herein for testing samples. At the end of this testing process, the values for the strain controls are recorded into LIMS 24. The magnitude of the signal and values provided by the positive control indicates the expected signal level for subsequently tested samples having the designated genetic sequence. The magnitude of the signal provided by the negative control indicating the expected signal level for subsequently tested samples that do not have the designate genetic sequence.

The computer at the screening laboratory 20 is configured to compare the test results (i.e. signal levels/values) for every sample that it subsequently tests for that designated genetic sequence with these multiple control signal levels and, based on that determination, to decide whether that sample has or does not have the designated genetic sequence. Positive and negative strain controls for a line therefore do not need to be resubmitted for each subsequent order but can be referenced by the screening laboratory 20 computer when later samples are tested for the same designated genetic sequence.

Upon receipt of the primers from a vendor, the sample, if available, will be screened using these reagents. Once a determination is made that there is discrimination between different genetic conditions, then the reagents will be placed in the inventory. Additionally, the screening laboratory 20 will populate a data field on the order management system, allowing the remote user 1 to select this primer sets or combination(s) for subsequent samples. This data filed will be populated with an indicator such as a mutation name, strain name or genetic line identification that will represent these reagents or combination of reagents that will be used in subsequent samples of this strain. This allows the remote user 1 to select the indicator of the reagents and prevents the need to transfer genetic information with each order.

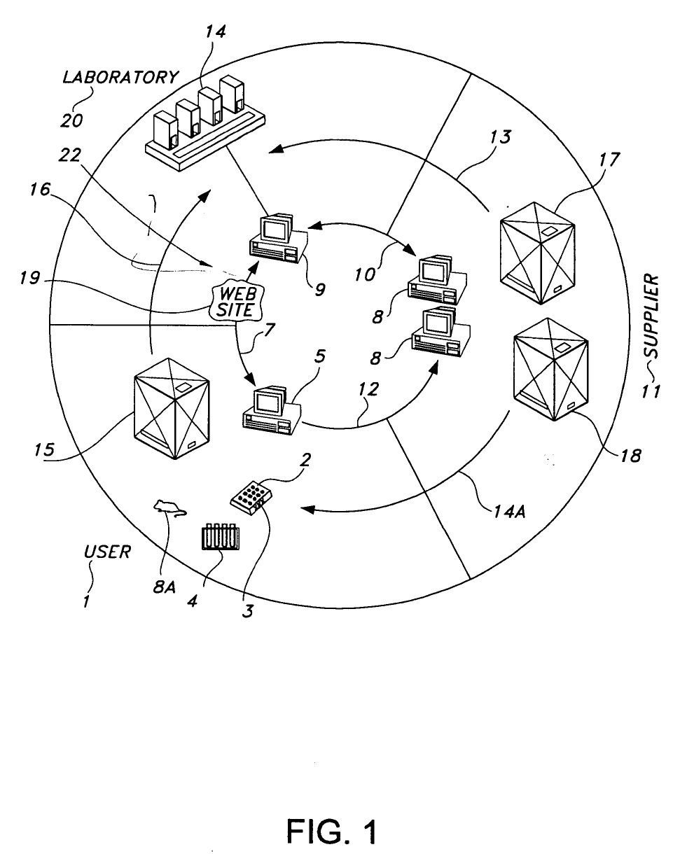

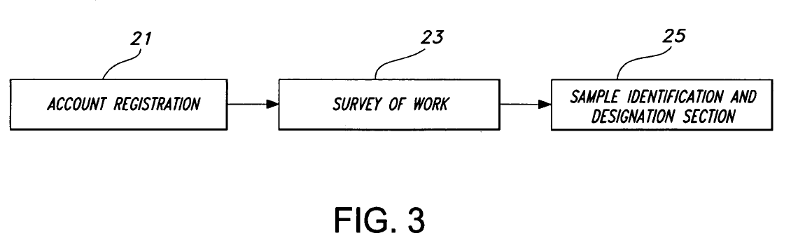

FIGS. 1-3 present an overview of certain features of the present invention. The present invention allows a remote user 1 with access to a computer 5 to order genotype screening of samples they submit to screening laboratory 20. Using the Internet or other communication link 7, the remote user 1 sends an access request from the remote user's computer 5 to a screening laboratory 20 computer 9 via an electronic communication link 7, such as the Internet. The screening laboratory 20 website 19 will transmit an access enabling response to the remote user 1 via electronic communication link 7. This response includes three distinct sections. The three sections are Account Registration 21, Survey of Work 23 and Sample Identification and Designation 25 (FIG. 3).

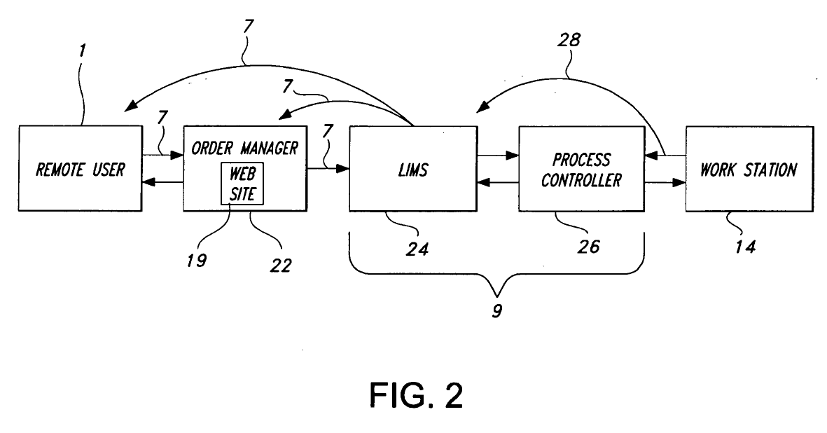

Now referring to FIG. 2, a remote user 1 can access screening laboratory 20 website 19 via communication link 7. The website 19 can be housed by an order manager 22. An order manager is a software-based order management system. In the preferred embodiment the order manager 22 is an order management system developed by “Big Fish”, a software development company in Memphis, Tenn. The order manager 22 functions to manage the placement of the order. The order received from the remote user 1 is transmitted to website 19, which reports the order to order manager 22. Manager 22 is in electronic communication via link 7 with screening laboratory 20 computer 9. Screening laboratory 20 computer 9 includes LIMS 24, which is communicatively coupled to a process controller 26.

LIMS 24 is the generic name for laboratory information management system software. The function of LIMS 24 is to be a repository for data, to control automation of a laboratory, to track samples, to chart work flow, and to provide electronic data capture. LIMS 24 can also, in another embodiment, be in direct communication with the remote user 1 via an electronic communications link 7. Any standard laboratory information management system software can configured to be used to provide these functions. Alternatively, a standard relational database management system such as Oracle (Oracle Corp., Redwood Shores, Calif.) or SQL Server (Microsoft Corp., Redmond, Wash.) either alone or in combination with a standard LIMS system can be used. In the preferred embodiment, the Nautilus® program (Thermo LabSystems, a business of Thermo Electron Corporation, Beverly, Mass.) is used.

The process controller 26 is communicatively coupled to the workstation 14. The process controller provides commands to any portions of the workstation 14 that are amenable to automation. For example, process controller 26 directs the delivery of the probes and primers to the Screening Station 95. The workstation 14 is communicatively linked 28 to LIMS 24. In this way, the workstation 14 can provide data to LIMS 24 for the formulation of the outcome report 249, and then, via link 7 to the order manager 22 or remote user 1. In an alternative embodiment, remote user 1 at remote user computer 5 can be linked 7 to the screening laboratory 20 by a direct phone line, cable or satellite connection.

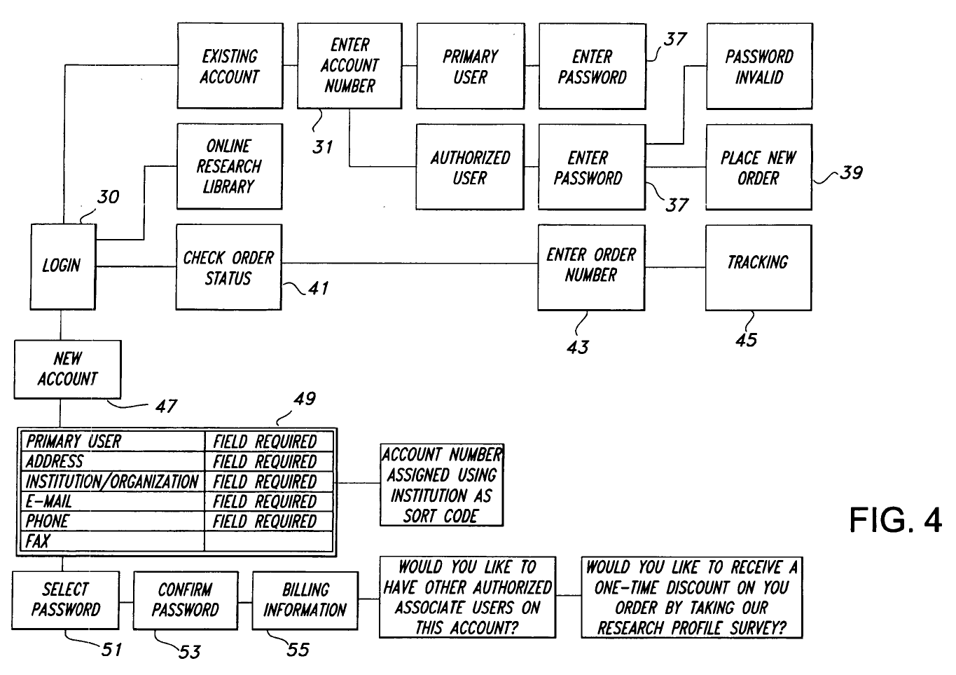

Now referring to FIG. 4, the user's Account Registration section 21 begins with logging into the system 30. A remote user 1 accesses an existing account by entering an account identification 31, which is, for example, an e-mail address. The user will then enter a password 37. If a valid password is entered, the user can place a new order 39. Alternatively, the user can check an order status 41 by providing an order number 43 and can proceed to order tracking 45. Alternatively, a new account 47 can be opened by providing an institution name, principal investigator, address, phone number, fax number, electronic mail address, billing information, and other authorized user names 49. The user can enter a password 51, confirm the password 53 and enter this billing information 55.

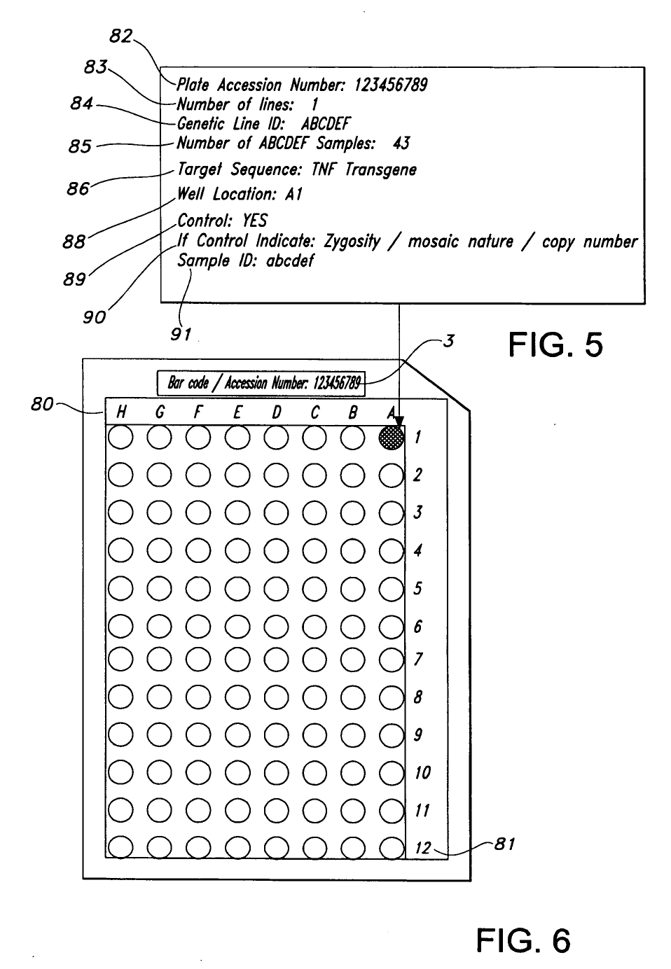

Now referring to FIGS. 5-6, once the remote user 1 submits the Survey of Work section 23 the remote user 1 will be presented with the Sample Identification and Designation section 25. In this section, the user (among other things) identifies where he will place each sample to be tested in an actual (physical) container 2 (FIG. 1) by associating each sample with a corresponding well of a virtual 96 well container displayed on the computer screen of computer 5 as described below. The Sample Identification and Designation section 25 includes 96 well container locations. The remote user 1 designates which sample was or will be placed into each well. If the remote user 1 has more than 96 samples, subsequent 96 source well containers and designations are available. With respect to FIG. 6, a 96 well source well container 2 having a barcode accession number 3 (FIG. 1) will be shown (FIG. 6) oriented in the longitudinal direction having an X axis labeled “A” to “H” (at 80) and a Y axis labeled “1” to “12” (at 81). The X and Y axes designate a well position such as “A1”.

FIGS. 5 and 6 together illustrate the Survey of Work section 23 and the Sample Identification and Designation Section 25. Referring now to FIG. 5, the remote user 1 is asked to provide: source well container 2 accession number 82, which the remote user 1 gets from the accession number 3 on the physical source well container 2 at his facility (FIG. 1) that he intends to fill (or has filled) with the samples, number of lines 83, genetic line identification 84, number of samples 85, and well location 88. The remote user 1 is also asked for any internal sample identification number 91.

For genotyping (i.e. screening to determine the presence of a designated genetic sequence) the positive strain control and the negative strain control samples may be designated and deposited in wells of a microwell container. The remote user 1 indicates that a sample is a control sample at 89. This assumes, of course, that the strain controls were not earlier provided to the screening laboratory 20 as described above.

At this point, the remote user has completed the Survey of Work section 23 and the Sample Designation section 25 of FIGS. 5-6 and is ready to transmit the screening parameter selections gathered in those sections to website 19 and thence to screening laboratory 20 computer 9.

Now referring to FIGS. 1 and 2, the remote user 1 transmits his or her order including the completed screening parameter selections to the screening laboratory 20 via link 7 such as the Internet or a direct line. The remote user 1 can transmit the selected screening parameter selections to LIMS 24 in screening laboratory 20 via electronic communications link 7. This link 7 can be direct or indirect. In the indirect route, the screening parameters are first transmitted to web site 19, wherein order manager 22 receives the order and then provides LIMS 24 with the screening parameter selections.

In a particularly preferred embodiment of the system described in the foregoing paragraphs, remote user 1 at computer 5 transmits a request for a home web page served by screening laboratory 20 web site 19 via the electronic communication link 7. Web site 19, in turn, serves a home web page to computer 5 that includes information identifying the source of the web page and including a login button. Remote user 1 at computer 5 clicks on the login button displayed on his computer screen, transmitting a signal to web site 19 requesting access to the web site. This request is transmitted over communications link 7 to web site 19, which responds with a second web page having fields for the entry of an account identifier (in the preferred embodiment an e-mail address), and a password. Remote user 1 enters the remote user 1 e-mail address and password, and transmits this information to web site 19 to gain access to the web site. Web site 19 receives this access request and compares the account identifier and password against its database of pre-existing accounts in the order manager 22 to determine whether the user is permitted to access the web site 19. If so, computer order manager 22 serves up a further web page, called an order manager web page, which includes several user selectable choices including an “order status” button for tracking previous orders and results (if any have been received), a “supply request” button for requesting supplies, and an “order” button for ordering additional tests.

To order genetic testing, user 1 clicks on the “order” button displayed on the screen of computer 5. Computer 5 transmits the user 1 request to web site 19. Web site 19 receives this request, and transmits a first ordering web page to computer 5. Computer 5, in turn, displays several fields on its computer screen, including several data entry widgets. The first of these widgets is list box including two selectable entries for requesting the speed of service. In the preferred embodiment there are two speeds of service: 24-hour service and 72 hour service. The second of these widgets is a list box providing several entries, each entry in the box corresponding to a profile/strain/ line identification 84 for which the sample is to be tested. The third widget is a text box for entering the number of samples of the selected strain to be tested. The fourth widget is a text box for entering the accession number (typically a bar code number) of the source well container 2 in which the samples are to be placed for shipping to the screening laboratory 20.

The remote user 1 types in the number of samples to be congenically screened. In this embodiment the samples are taken from transgenic animals on a C57BL/6 background, each sample typically corresponding to one animal to be tested. Typically several animals are tested to determine if they received the genetic background from their recipient parents. Each strain of animal is defined by one or more designated genetic sequence. Thus, by designating the strain for which the samples are to be tested, the remote user 1 selects the one or more designated genetic sequences associated with that sequence. In the preferred embodiment, the remote user 1 can also select or deselect each individual primer set that is used to screen for the designated sequences in the strain/line/profile of the biological sample.

Once the remote user 1 has entered the number of samples to be tested, he or she then enters the name of the strain identification that the samples are to be tested for. Again, by selecting a strain the remote user 1 indicates the designated genetic sequence for which the samples are to be tested, since each strain is bred to have that sequence.

Once remote user 1 has selected the speed of service, the strain to be tested, and the number of samples to be tested for that strain, he enters the accession number from the source well container 2 and clicks on a button on the first ordering web page for recording this first group of samples to be tested. Computer 5, in turn, generates a revised first ordering web page, the revised page including a table entry in a table on the revised web page listing the first group of samples in tabular form, wherein each row in the table corresponds to one group of samples to be tested, identifying that group of samples by the strains for which that group of samples is to be tested, and the number of samples in that group.

This process of creating a new group of samples and identifying them by the strain for which they'll be tested, and the number of the samples, can be continued as many times as necessary until all the samples to be tested are identified in the table.

Once all of the groups of samples have been entered and listed in the table on the revised first ordering web page, the operator then selects a button identified “next” and moves to the next stage in the ordering process. Computer 5 transmits this request to web site 19, which generates a graphical image of a 96 source well container, appearing on the screen of computer 5 identical to the corresponding 96 source well container 2 that the remote user 1 is filling/has filled with samples, and transmits that image embedded in a second web page back to computer 5 for display. The second web page includes a graphical representation of a 96 well plate, in a top view, showing the two dimensional array of all 96 wells in which the remote user 1 is to place the samples identified previously. Web site 19 calculates the respective positions of each group of samples in the well container 2. Each group is shown in the graphical representation of the well plate in a different color. All the wells in a group are shaded with the color associated with that group.

Samples of the same color from the same group are grouped together thus producing several different contiguous groups of wells, each group of wells have the same color different from the color of the adjacent groups.

The images of the wells in the web page are displayed on the computer with an initial shading to indicate that they have not been identified to a particular animal from which the sample in each well will be taken. In the preferred embodiment, each well contains a sample, such as a tissue sample, taken from an individual animal. The purpose of the testing performed on the samples in the wells is to determine the genetic characteristics of the animal from which each sample was taken. In order to relate the test results performed on each sample back to the animal from which the sample was taken, the user must make a record of the animal source of each sample (i.e. the animal from which each sample was taken).

To uniquely identify each sample in each well with an associated animal, remote user 1 selects a button on the third ordering web page. This button signals computer 9 to generate an additional web page. This web page lists each well in the well plate that was previously identified as containing a sample. Thus, if the first group of samples were 13 in number, there would be 13 entries listed in the additional web page. The web page itself is arranged as a single column of entries. Each entry in the column of entries includes a well identifier (called well location 88, above), which is a string of alphanumeric characters that uniquely identifies one well of source well container 2. A preferred well identifier for the 96 well plate is an alphabetic character followed by a numeric character. A text box is adjacent to each well identifier on the additional web page. To uniquely identify each sample in the source well container 2, the user enters alphanumeric characters in the text box that are uniquely associated with each sample. This identifier is typically a short string of consecutive alphabet or numeric characters, a practice commonly used by research facilities to identify individual animals used for testing.

Animals in a particular group of animals having (presumed) common genetic characteristics will typically be identified by tattoos, tags, or other permanent means by consecutive or sequential numbers, characters, or combinations of numbers and characters (for example “A1”, “A2”, “A3”, or “101”, “102”, 103”, or “AA”, AB”, “AC”, etc.). In a preferred embodiment, user 1 enters each animal number into the text box as a sample ID 91. Animals may also be identified by a unique combination of disfigurements such as cutting or cropping toes, tails or ears that can also be approximated to a progressive alphanumeric sequence.

To assist the remote user 1 in entering the sample ID 91 into each of the text boxes in the additional web page, a button is provided to automatically fill several consecutive text boxes based upon the alphanumeric characters typed into a few text boxes from the group. For example, if the user types in “B7” in the first text box of a group, then types in “B8” in the second text box of a group, computer 5 is configured to automatically generate consecutive alphanumeric strings to fill the remaining text boxes of the group based upon these two manually typed-in entries. In this case, computer 5 would automatically generate the alphanumeric strings “B9”, “B10”, “B11”, etc. and insert these characters sequentially into the remaining text boxes of the group in the additional web page. This process can be repeated for each subsequent group shown on the additional web page. Alternatively, the computer can be configured to automatically generate alphanumeric characters for all the groups at once and to fill the text boxes of all the groups all at once. Once the user has finished identifying all of the groups of samples and filling out all of the sample ID's 91 in the text boxes on the screen of computer 5, he clicks on a button labeled “next”. Computer 5 transmits this request to website 19, which responsively generates another web page in which the user 1 enters shipping and tracking information. This page, called the order confirmation page, includes a text box for entering a character string. This character string provides access to a web-based shipment tracking system of a commercial shipping company. In the preferred embodiment, the character string is a tracking number used by the shipping company to track the samples from the remote user 1 to the screening laboratory 20. In the preferred embodiment, the tracking number is provided to the user together with the source well container 2 and the packaging materials in which the user places the source well container 2 for shipment to the screening lab 20.

The order confirmation page also includes an invoice that lists the different tests requested by the operator in the foregoing steps on the screen of computer 5. Each test or group of tests is displayed on the screen adjacent to the price or prices for those tests. A total price of all the tests is displayed as well.

The order confirmation page has a second text box in which the remote user 1 can type the expected shipping date. The expected shipping date is the date on which remote user 1 intends to give the samples in their packaging materials to the delivery service associated with the tracking number. By providing the anticipated shipping date to the website 19 and then to the screening laboratory 20, personnel at the screening laboratory 20 can anticipate the arrival of each shipment and prepare for its arrival by pre-ordering reagents and primer sets required for testing the samples in advance.

Once the operator has entered the tracking number and the expected shipping date, he clicks on a button labeled “confirm order”, which transmits the completed order, including the tracking number and expected shipping date to website 19 and order manager 22, and thence to LIMS 24.

In the preferred embodiment, once the order has been transmitted to the order manager 22, the order generates two electronic messages, which will be sent to different locations. The first message is cross-referenced in LIMS 24 with a list of stocked primers. If the primer set designated by the user is not stocked, an order message is sent to a supplier 11, such as a contracted probe provider. This request can be transmitted from remote user 1 to screening laboratory 20 via any form of electronic communication, and then via a form of electronic communication 10 to suppliers' computer 8, or in the alternative, the order message can go from user 1 via any form of electronic communication link 12 to suppliers' computer 8. The supplier 11 creates the primer sets based on the designated genetic sequence designated by the remote user 1 or the screening laboratory 20. This supplier 11 will then barcode and overnight ship 13 the primer sets to the screening laboratory 20. Once the primer sets for each order for that day's screening are received by screening laboratory 20, the barcodes on the primer sets are scanned into LIMS 24. The LIMS 24 records the date and time the primers were received along with the quality control data provided from the primer provider.

In the preferred embodiment, the primer sets are placed in workstation 14 and LIMS 24 will record the barcode of the primer and record its specific location on the deck of the workstation 14, as will be discussed in more detail with respect to the Screening Station 95. Additionally, the screening laboratory 20 and the LIMS 24 system correlates which primer sets will be used on which samples, as will be discussed in more detail with regard to the Screening Station 95.

The second message, in the preferred embodiment, that is generated from the order placement of the remote user 1 insures that the remote user 1 has the proper supplies to package and ship their samples. This message, sent via link 12, will define the barcode number of well container(s), shipping labels tracking number and amount of reagents needed for the user. In response to this message, supplier 11 will package 18 supplies for remote user 1 and ship 14A the supplies back to remote user 1.

Once the remote user 1 procures or receives these supplies, the remote user 1 places the appropriate samples into the source well containers 2 previously identified in the order sent to website 19, order manager 22 and LIMS 24. In other words, the remote user 1 fills each well of source well container 2 such that each well contains the same sample with the same sample ID 91 that the user previously identified in the order previously sent to website 19. Alternatively, if the user already had sufficient supplies when the user placed the order the user need not wait for a source well container 2 to be sent by a supplier, but can fill the source well container 2 when the user creates the order, or even before the order is created. What is important is that the contents of the actual 96 source well container 2 that the user fills exactly matches the description of the samples and has the same accession number as the order the user previously sent to website 19.

The samples can be obtained from prokaryotic or eukaryotic organisms. The samples may be a tissue sample, swabs or other biological biomatter such as blood, semen, or lymph from a mouse 8A, but can also come from other animals (including humans), plants and viruses. In the preferred embodiment, mouse tails or ears are snipped to provide a tissue sample. Source well container 2 is a 96 well plate or the like that receives the sample in each well of the well plate. A sufficient amount of lysis reagent can be added to cover the sample. In one embodiment, the lysis reagent is added prior to transit to the screening laboratory 20. Although, in the preferred embodiment the lysis reagent is added at the screening laboratory 20 at Lysing Station 92.

A biological sample can be collected in a variety of ways to facilitate rapid screening. In one embodiment, the collection method involves swabbing the oral, nasal or anal cavity of an animal to be tested, such as a mouse, to collect cells for screening. In this collection method swab tips are removed by the remote user 1 and placed in individual wells of a multi-well container for transport to the screening laboratory 20. Many different swab materials may be used including polyester, cotton, acrylamide, nylon and calcium alginate. In the preferred embodiment Microbrush® (Graftin, Wis.) nylon swabs are used. A multi-well container as shown in FIG. 1, in the preferred embodiment, is a 96 microwell source well container 2 but can include other multi-well containers, such as Strip Racks, 24 well plates, 384 well plates and tube rack holders or the like. As described above with regard to FIG. 6, the remote user 1 operates computer 5 to enter a variety of data regarding the samples placed in the source well container. Once all of the samples in all of the wells have been identified in this manner, the remote user sends the source well container 2 containing a plurality of biological samples to a screening laboratory 20 for screening.

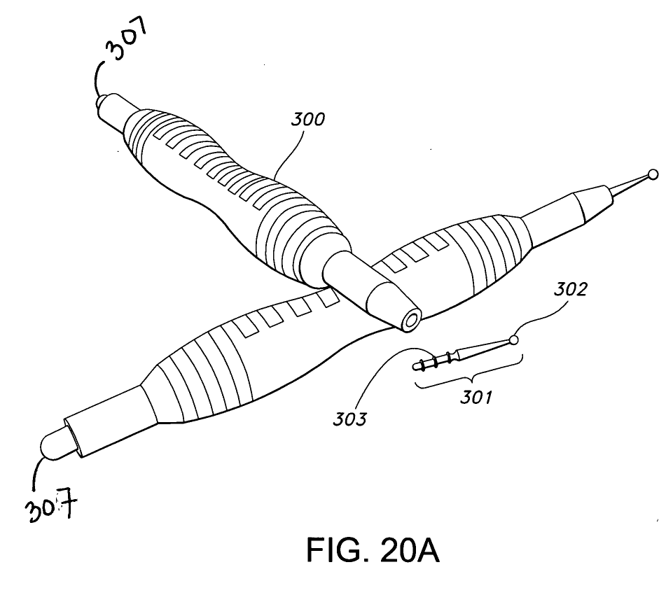

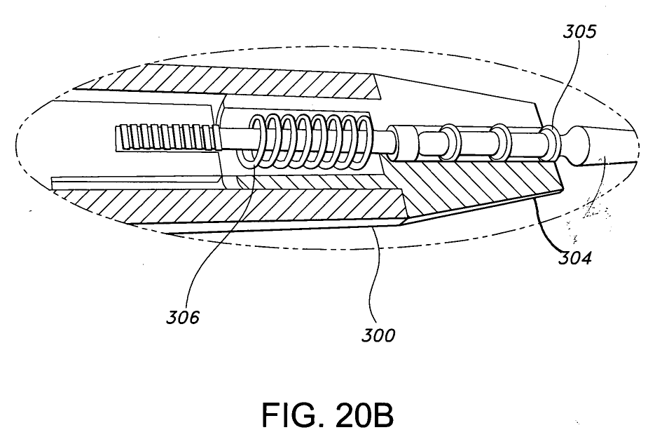

Now referring to FIG. 20A and 20B, an apparatus to swab the subject and to facilitate placement of the swab into a source well container 2 is disclosed. A swab holder 300 with disposable swab 301 is shown. The swab 301 has a proximal and a distal end with respect to a swab holder 300. The distal end of the swab 301 is made of a sufficient amount of flocking to collect a biological sample. The proximal end of the swab 301 has at least one annulus 305. The function of the at least one annulus 305 is to secure the swab 301 to the swab holder 300 during swabbing of a subject. The swab holder 300 has an internal section configured to retain at least one annulus of a swab 301. In the preferred embodiment, the internal section 304 is deformable. This section can be elastomeric, serving as a swab grip, which receives and holds the disposable swab 301 until released by the spring plunger 306. In the preferred embodiment the mounting end of the swab 301 tip has at least one annulus 305 which, upon insertion into the swab grip, deforms or squeezes into the elastomer sufficiently to retain the swab 301 during its function. Although three annuli are shown in the FIG. 20A, it would be possible for one elongated annulus (not shown) to serve the purpose. A spring loaded plunger 306 has a release button 307 on opposite end from swab 301. The action is like that of a retractable ball point pen but without the latch function. The swab holder 300 preferably includes an elastomeric, rigid plastic grip area, metal or the like on outer surface with metal, metallized plastic or the like main body.

In the preferred embodiment, a swab 301 is made of a plastic material that measures approximately 1 inch long with a diameter of approximately 0.050 inches. The distal portion of the swab 301 is flocked with nylon fibers. Whereas, the proximal end of the swab 301 shaft is designed to fit into the swab holder 300.

After the swab 301 is seated in the swab holder 300 the remaining portion of the swab 301 shaft and flocking are inserted into an orifice of a subject to collect biomatter. The swab 301 and/or swab holder 300 may be rotated to facilitate the collection of biomatter. The body of the swab holder 300 is linear with respect to the swab 301 to facilitate collection of biomatter. Upon sufficient collection of the biomatter, a mechanism 307 is depressed on the swab holder 300, such as a button that ejects the swab 301 from the distal end of the swab holder 300. The ejector mechanism is then loaded with a new swab 301 and the process is repeated as many times as necessary.

In another embodiment of this invention, the biological sample is a blood sample collected by nicking the animal to be tested and blotting the blood on a filter paper. The blotted filter paper is placed in individual wells of source well container 2 by the remote user 1 and transported to the screening laboratory 20. In both of these embodiments, the biological sample is disposed on an absorbent carrier.

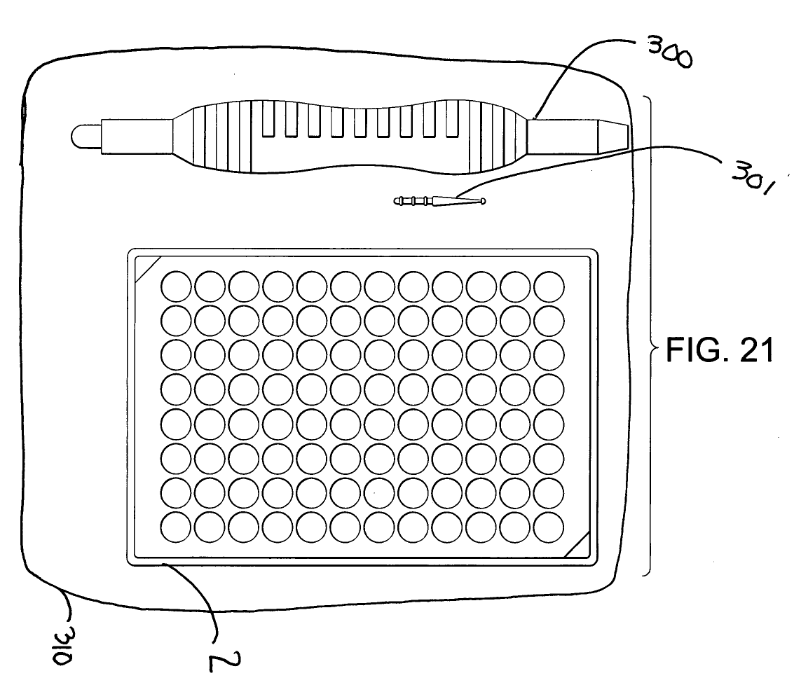

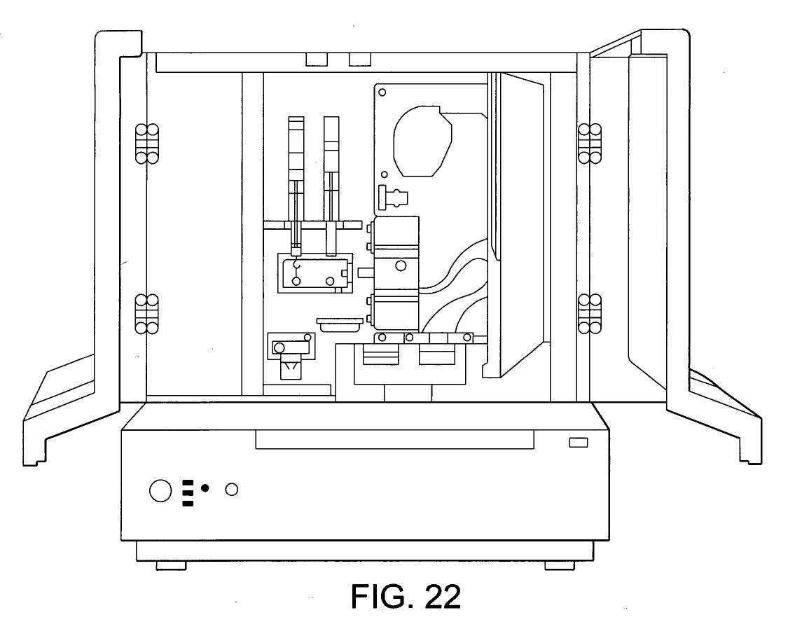

Now referring to FIG. 21, the swab holder apparatus 300, swab 301 and a source well container 2 can be packaged in a kit and sent to a remote user 1. The kit 310 does not need to be sterilized.

Referring now to FIG. 1, source well container 2 has an accession number 3 affixed to the side of the container. The accession number is used by LIMS 24 to track the source of source well container 2. The remote user 1 places the appropriate samples into the well locations in source well container 2 that they had previously designated while placing their order in FIG. 6. The remote user 1 will add lysis reagent 4 to each well of the source well container 2. The lysis reagent 4 should cover the samples. Once the samples and lysis reagent 4 are in the source well container 2 the remote user 1 places a seal on the top of the source well container 2 preventing samples from leaking. The remote user 1 then places a plastic lid on the seal for transportation. The remote user 1 then places the source well container 2 into an overnight delivery service package 15. The remote user 1 will then seal the package and ship 16 to screening laboratory 20, and apply a barcode shipping label.

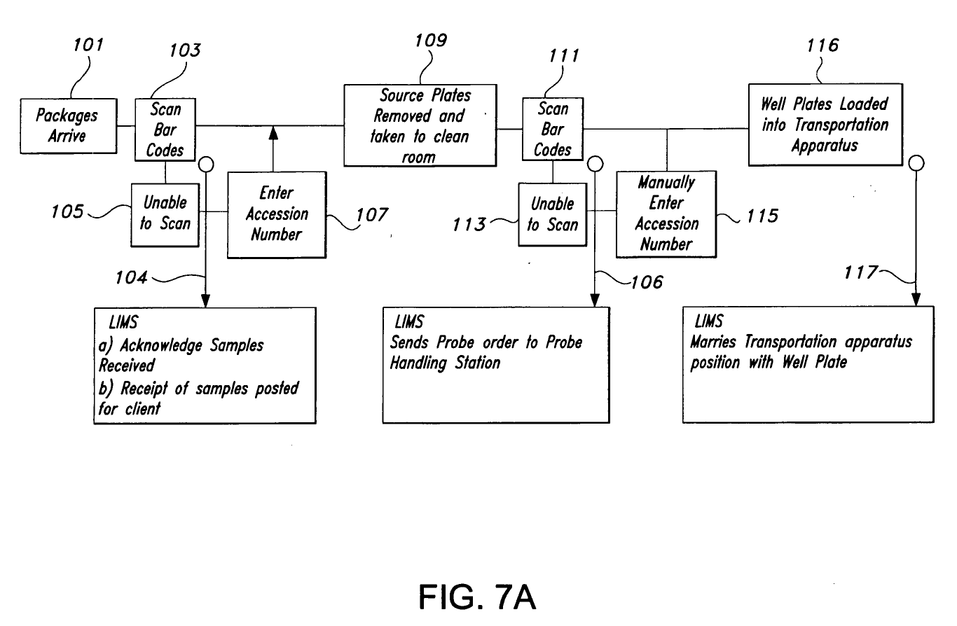

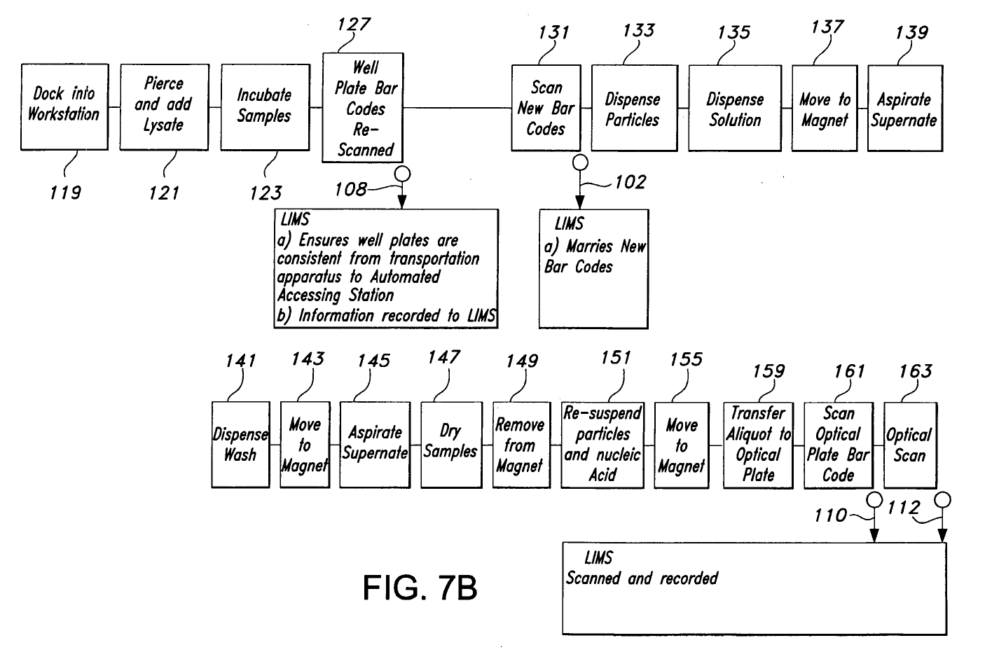

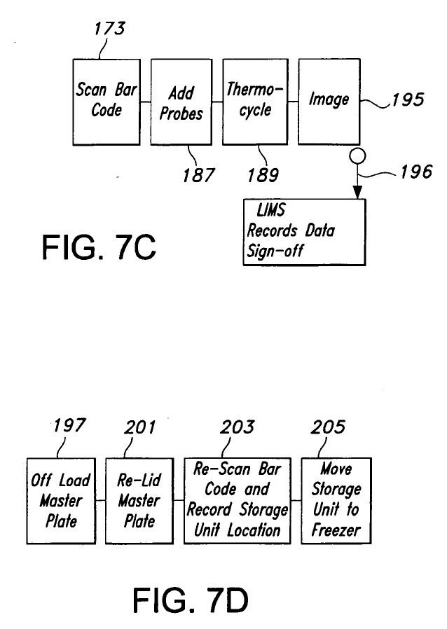

Now referring to FIG. 7A-D, the preferred embodiment of the present invention is shown. In FIG. 7A, the source well containers 2 arrive 101 at the screening laboratory 20. The tracking number of the shipping label is read with a barcode reader 103. If the shipping label is unreadable 105, the tracking numbers are manually entered 107. The scanning of the tracking number is received 104 in LIMS 24 and a received message is posted to the user's account as shown in tracking field. The source well container 2 are removed from the package and taken to a clean room 109. The source well containers 2 contain the raw biological matter and in one embodiment lysis reagent. The source well containers 2 individual barcodes are scanned by the barcode reader 111 and recorded 106 in LIMS 24 as accession numbers. LIMS 24 can send 106 a primer set order to supplier 11 through the order manager 22. If the source well containers 2 individual barcodes are unable to be scanned 113, the accession numbers are entered manually 115. If the tracking number, accession number, user order and worklist properly correlate, LIMS 24 will activate (not shown) an active record number for the containers.

The source well containers 2 are loaded 116 into a transportation apparatus in a clean room. A transportation apparatus is any device that holds well containers and that can dock with the workstation. The transportation apparatus, in the preferred embodiment, includes several rigid trays stacked vertically in a housing unit that is mobile. This transportation apparatus can be moved between different automated stations, docked and the rigid trays can be removed in an automated fashion and processed on the deck of a workstation. Each rigid tray consists of nine locations for source well containers 2. Each of these nine locations per tray has a unique barcode designating its specific location inside the trays of the transportation module.

Source well container 2 accession number 3 is scanned with a barcode reader and the bar-coded source well container 2 location in the transportation apparatus trays is scanned. The barcodes of source well containers 2 are married 117 in LIMS 24 with the unique barcode locations in the transportation apparatus trays for tracking purposes. LIMS 24 records and associates each well container to this location. Once the transportation apparatus is loaded with the source well containers 2, the transportation apparatus is docked 119 into the laboratory workstation 14.

LIMS 24 will generate a worksheet for laboratory personnel (not shown). The worksheet outlines the primer sets that the operator will need to prepare or gather in order to test the latest samples. The LIMS 24 worklist will generate a single file. The file format may include, but is not limited to, ASCII, XML or HTML. The file will be written into a specified directory on the network drive. The name of the file will be unique and will correlate to a run number. The extension will be unique for worklist files.

In the configuration described above, a transportation apparatus includes a housing unit provided to support several trays, each tray having nine different locations for nine source well containers 2. In an alternative embodiment, however, the housing unit can be eliminated. Instead, the source well containers 2 can be manually transported throughout the workstation in trays from functional station to functional station. In this system, operator at the laboratory loads source well containers into the trays after the source well containers 2 are received at the screening laboratory 20 and are scanned into LIMS 24 as described above for transportation to workstation 14. Alternatively, source well containers 2 can be transported individually to workstation 14 and be placed in a tray or trays that are already located at workstation 14.

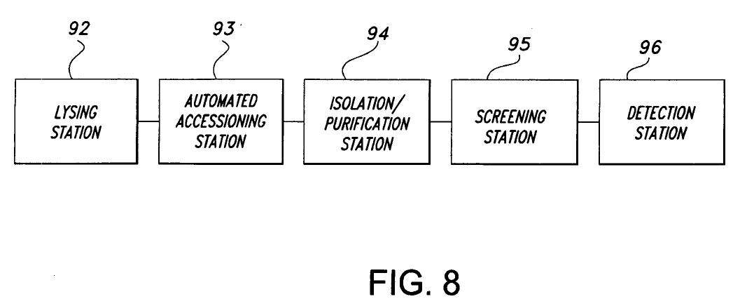

We now refer to FIG. 8, which depicts one embodiment of the workstation 14. Standard laboratory stations are logical groupings of laboratory operations. These groupings, however, do not necessarily refer to different physical stations. These logical groupings include: Lysing Station 92, Automated Accessioning Station 93, Isolation/Purification Station 94, Screening Station 95 and Detection Station 96, all of whom make up the workstation 14. The Screening Station 95 can include other screening processes such as PCR. Lysing Station 92 is an alternative step provided to lyse the samples in containers 2 in the event user 1 does not choose to lyse the samples by adding a lysis reagent before sending them to laboratory 20. The functions of the various logical stations are described below in connection with the steps shown in FIGS. 7A-D. The following description provides the preferred embodiment, although one skilled in the art could elect to conduct these methods with varying degrees of automation as required.

As mentioned above, remote user 1 need not add a lysis reagent to the samples before shipping them to screening laboratory 20. Instead, the samples may be shipped un-lysed (at room temperature if tissue, frozen if swabs) and may be lysed at laboratory 20 by piercing the cover 121 of the container 2 and treating each of the samples with a lysis reagent after docking the tray in the workstation 119 in the lysing station 92. The samples are incubated 123 to produce a lysate containing cellular debris including at least a portion of intact genomic nucleic acid.

With respect to the swab sample collection method, a sufficient amount of a lysis reagent, such as SV Lysis reagent or Nucleic Lysing Solution (Promega Corporation, Madison, Wis.) is added to each well of source well containers 2 to cover the swab tips. Swabs do not need to be incubated for three hours, however they may be vortexed for ten minutes.

With respect to the blood sample collection method, a sufficient amount of a lysis reagent, such as Nuclei Lysing Solution (Promega Corporation, Madison, Wis.) is added to each well of source well containers 2 to cover the filter paper. With respect to animal embryonic tissue, stem cell screening and tissue biopsies Nuclei Lysing Solution (Promega Corporation, Madison, Wis.) is added to each well containing the tissue. The source well container 2 is treated under conditions to facilitate rapid lysis of the biological sample. In the preferred embodiment, these conditions are heating at 55° C. for three hours.

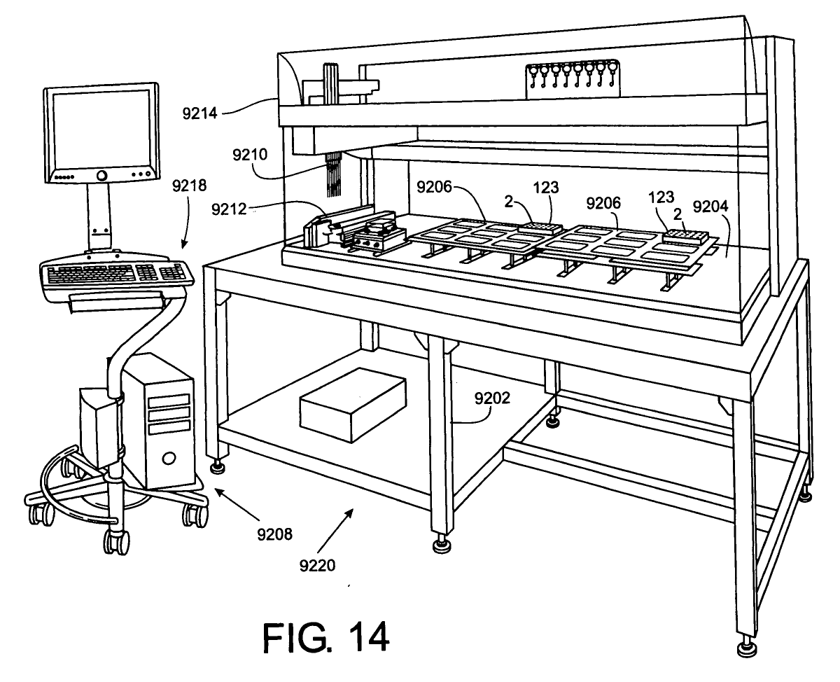

The preferred method of performing the above lysing steps at Lysing Station 92 includes loading source well containers 2 into the tray 9206 and taking the rigid tray to Lysing Station 92 to be lysed. Lysing Station 92 includes a liquid handler 9220, such as Genesis Tecan (Raleigh Durham, N.C.) or Multimeck Beckman (Indianapolis, Ind.). An example of a preferred Lysing Station 92 is shown in FIG. 14. It includes a frame 9202, on which a deck 9204 is mounted to provide a horizontal working surface, which supports tray 9206, which supports and positions up to nine source well containers 2. A material handler 9214 is fixed to frame 9202 and extends upward and across the top surface of deck 9204. A computer 9208 is coupled to material handler 9206 to direct the movement and operation of pipettes 9210. A trough or reservoir 9212 is provided on deck 9204, from which computer 9208 commands the material handler 9214 to aspirate lysis reagent into pipettes 9210 and to deposit the reagent into wells of container 2.

The operator first carries a plurality of source well containers 2 and places them on deck 9204 in one of the nine positions on the rigid tray 9206 that support and orient source well containers 2 thereby docking them 119 into the workstation 14. The operator then enters the number of wells that are filled with samples in each of the source well containers 2 into computer 9208 in combination with the location of that container with respect to tray 9206.

Knowing the location of each source well container 2 in tray 9206, and the number of wells that are filled with samples in each of these source well containers 2, computer 9208 then directs material handler 9214 to move the pipettes 9210 to each source well container 2 in turn, piercing 121 the barrier sealing mechanism and filling each of the wells of source well containers 2 containing a sample with lysis reagent. By providing the location and the number of samples, computer 9208 is configured to fill only the wells containing samples with lysis reagent and to leave the empty wells empty of lysis reagent.

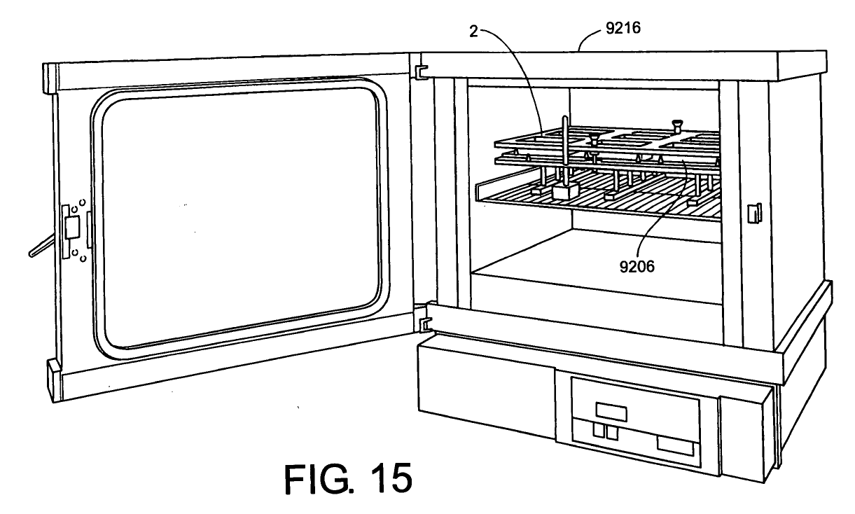

Once each of the sample-containing wells has been filled with lysis reagent, the operator moves the entire tray or trays 9206 containing the samples to an oven 9216 (FIG. 15), where the samples may or may not be incubated 123 by heating for a period of about three hours at a temperature of 55° C. (described above) depending on the sample type. Once the incubation process is complete, the operator moves source well containers 2 supported on the tray or trays 9206 to Automated Accessioning Station 93.

An Automated Accessioning Station 93 provides a device to remove liquid from the source well container 2 to the primary master well container 6. The primary master well container 6 is the container in which the nucleic acid is isolated. It is preferably a 384 well plate (Fisher Scientific #NC9134044). Any commercially available automated accessioning device can perform this fumction such as Genesis® Tecan (Raleigh-Durham, N.C.) or Multimeck® Beckman (Indianapolis, Ind.). These devices are referred to as liquid handlers. The source well containers 2 barcode accession numbers 3 are re-scanned 127. This measurement will be recorded and posted 108 into the LIMS 24 database and reflected in the outcome report 249. Additionally, LIMS 24 ensures 108 that source well containers 2 are consistent from transportation apparatus to the Automated Accessioning Station 93. Error codes will be generated if a sufficient amount of raw testing material is not available. The liquid handler utilizes stainless steel, or the like, pipette tips that are washed between each sample transfer. Alternatively, disposable pipette tips may be used.

The nucleic acid lysate is transferred 129 to clean well containers, called primary master well containers 6. Each of the containers 6 has a scannable accession number, preferably a barcode accession number, called “barcodes” or “accession numbers” below. The barcodes of the primary master well containers 6 are scanned 131 and LIMS 24 marries 102 the barcodes for the primary master well containers 6 to the scanned barcode accession numbers 3 of the source well plates 2. The automated process accessioning continues until all of the day's pending samples are accessioned into the primary master well containers 6. The preferred method of performing the above steps at Accessioning Station 93 includes taking the rigid tray 9206 and the source well containers 2 from the incubating oven 9216 back to the same liquid handler 9220 that performs the functions of Lysing Station 92. This liquid handler 9220 is also preferably configured to function as Accessioning Station 93.

Referring now to FIG. 14, the operator returns tray 9206 to liquid handler 9220 and places tray 9206 back on deck 9204 generally in the same location it was in when the lysis reagent was inserted into each well containing a sample.

Once in that location, the operator commands computer 9208 to fetch the work list from LIMS 24 and electronically stores it in the computer memory of process controller 26. The work list includes the accession numbers of each source well container 2 that is in tray 9206, together with the primer sets that should be used for each well. The work list uniquely associates the location of the well, the accession number of source well container 2 from which the well is from, the probe type that is to be used with the sample in that source well container 2, and the quantity of primer to be added to that sample.

Once computer 9208 fetches the work list, computer 9208 directs the operator to electronically scan 127 the accession numbers of all the source well containers 2 that are in rigid tray 9206 on deck 9204 of liquid handler 9220 using scanning device 9218 coupled to computer 9208. Scanning device 9218 is preferably a glyph scanner, character scanner, bar code scanner, dot matrix scanner, or RFID tag scanner, depending upon the form of the accession identifier (typically a barcode accession number 3) on source well container 2. Once source well containers 2 have been scanned 127, computer 9208 transmits 108 the accession numbers 3 to process controller 26 and thence to LIMS 24. Process controller 26 preferably includes an instrument database to which each of the computers of Lysing Station 92, Automated Accessioning Station 93, Isolation/Purification Station 94, Screening Station 95 and Detection Station 96 transmit their data in order to maintain an ongoing record of the testing process and the location of materials and samples throughout that process. The database is preferably implemented using Microsoft's SQL Server, although any relational database (e.g. Oracle), may be used.

Computer 9208 then commands material handler 9206 to transfer 129 the contents of each well (i.e. lysate) in source well containers 2 to a corresponding well in the primary master well container 6 using pipettes 9210. Computer 9208 directs the operator to scan 131 the accession numbers on the primary master well container 6. Like the accession number on source well containers 2, the accession number on the primary master well container 6 may be any electronically scannable indicia or device. Computer 9208 transmits the accession numbers to process controller 26, which sends them to LIMS 24. In this manner, LIMS 24 maintains a record of each sample and its location in each source well container 2 and in each primary master well container 6. LIMS 24 and process controller 26 correlate the accession number of each primary master well container 6 with the identity of each sample it contains, the strain/line/profile for which each sample is to be tested, the designated genetic sequence or sequences that identify or indicate that strain and primer sets necessary to test for those designated genetic sequences and the results of the testing.

The tray of primary master well containers is moved by the transportation apparatus to the Isolation/Purification Station 94. In this station, the genomic nucleic acid will be isolated and purified using a separation method such as magnetic or paramagnetic particles. Purified genomic nucleic acid, substantially free of protein or chemical contamination is obtained by adding a sufficient amount of magnetic particles to each of the well containers that bind to a predefined quantity of nucleic acid. The term “magnetic” in the present specification means both magnetic and paramagnetic. The magnetic particles can range from 0.1 micron in mean diameter to 100 microns in mean diameter. The magnetic particles can be functionalized as shown by Hawkins, U.S. Pat. No. 5,705,628 at col. 3 (hereinafter '628 patent hereby incorporated by reference).

In the preferred embodiment, the magnetic particles are purchased from Promega Corporation, a measured amount of magnetically responsive particles are added 133 to the lysate mixture with or without the presence of a chaotropic salt 135. In the preferred embodiment, 13 μl amounts of 1 micron silica magnetic particles with chaotrope 113 μl (Promega Corporation, Madison, Wis.) are added to each well of the microwell container. The fixed volume of particles may becomes saturated with nucleic acid and excess nucleic acid is removed. It has been observed that the resulting nucleic acid concentration between samples is very consistent. In a 50 μl pathlength read by the Geriios (Tecan, Research Triangle Park, N.C.) a standard A₂₆₀ is 0.2 OD units. A standard concentration range of 0.1 to 0.3 O.D. units is disassociated from the magnetic particles to yield purified genomic nucleic acid for tissue biopsies.

Table 1 shows that with increasing amounts of magnetic particles, the nucleic acid concentration also increases when excess nucleic acid is present in the lysate.

TABLE 1

| | | Bead Volume per |
|---|---|---|
| Average | Stdev | 150 μl of lysate |
| 0.7974 | 0.0072 | 27 |
| 0.8750 | 0.040 | 35 |
| 1.2328 | 0.026 | 50 |
| 1.7900 | 0.022 | 75 |

While the nucleic acid concentration may be consistent between samples treated with the same protocol, several factors may increase or decrease the resulting standard concentration of genomic nucleic acid. These factors include: amount of nucleic acid in the lysate, the binding reagent, the number of purification washes, and the solution that is used to elute the nucleic acid. The preferred binding solution for the magnetic particles obtained from Promega (Madison, Wis.) is a chaotropic salt, such as guadinium isothiocyanate. Alternatively, other binding reagents, such as 20% polyethylene glycol (PEG) 8000, 0.02% sodium azide and 2.5M sodium chloride may be used to nonspecifically bind the genomic nucleic acid to the surface chemistry of the functionalized magnetic particles. If finctionalized magnetic particles are used, the preferred binding solution is PEG. The PEG or chaotropic guadinium isothiocyanate allows for the disruption of hydrogen binding of water, which causes binding of the nucleic acid to the particles. The preferred washing procedure to remove contaminants includes two chaotrope washes, after the initial chaotrope binding step, followed by four 95% ethanol washes. Aqueous solutions, or the like, are the best elution solutions. These solutions include water, saline sodium citrate (SSC) and Tris Borate EDTA (ie. 1×TBE).

The amount of DNA isolated from the swabs and blood is less than the DNA yield recovered from tissue. The tissue lysate has enough DNA content to saturate the binding ability of the fixed volume of beads. However, the swab and blood lysate does not have enough DNA to saturate the binding ability of the fixed amount of beads. This is evidence by Real-Time PCR CT (cycle threshold) values for the housekeeping probe. The housekeeping (cjun) CT values for tissue isolations are approximately 26 whereas the approximate CT for housekeeping (cjun) for the blood isolations are approximately 35. This nine cycle difference represents approximately a 512 (2\hat{ }9) fold difference in the amount DNA present. This nonsaturated DNA yield does not present a problem for results because the detection of the designated genetic sequence can be done on the pictogram scale.

The preferred device for performing the above functions of the Isolation/Purification Station 94 is a liquid handler 9402 identical in general construction to the liquid handler 9220 identified above for use as the Lysing Station 92 and the Accessioning Station 93 that has been configured to automatically transfer the various reagents and other liquids as well as the magnetic particles in the manner described below.

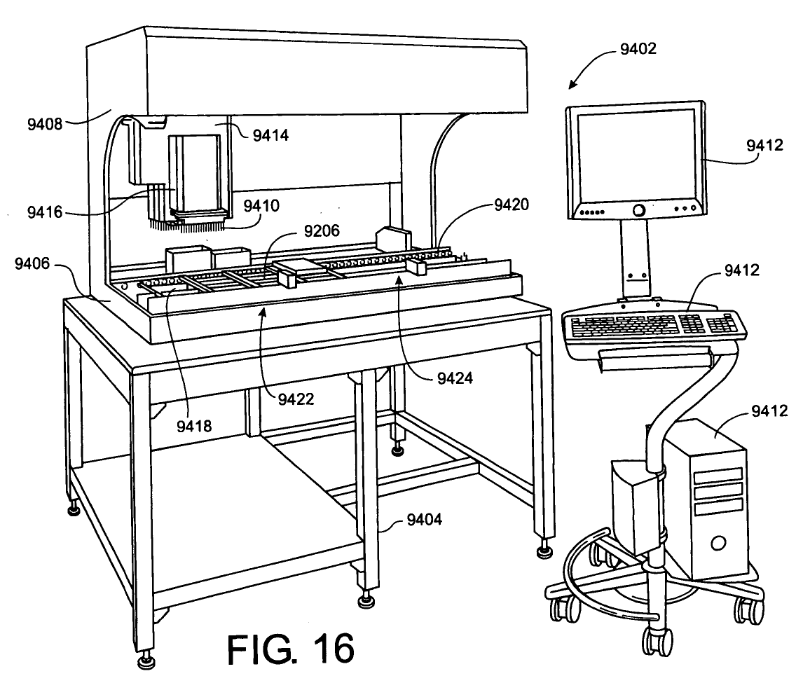

FIG. 16 illustrates a preferred embodiment of the liquid handler 9402. Handler 9402 comprises a frame 9404 on which is mounted a deck 9406, which is surmounted by material handler 9408, which supports and positions pipettes 9410 and is coupled to and controlled by computer 9412, which is in turn coupled to process controller 26 to communicate information to and from LIMS 24. Liquid handler 9402 includes a syringe pump 9414 that is coupled to and driven by computer 9412 to dispense magnetic particles via a 16×24 array of 384 pipettes 9410 simultaneously into all 384 wells of the primary master well container 6 under the command of computer 9412. Liquid handler 9402 also includes a second syringe pump 9416 that is configured to dispense a binding buffer into wells of the primary master well container 6 under computer control. The liquid handler also includes a magnet 9418 mounted in deck 9406 as well as a conveyor 9420 that is coupled to and controlled by computer 9412 to move the primary master well container 6 in tray 9206 back and forth between a first position 9422 in which the container is within the magnetic field and a second position 9424 in which the container is outside the magnetic field.

Before the functions of the Isolation and Purification Station 94 can be performed, the operator must first move the primary master well container 6 from Accessioning Station 93 to deck 9406 of liquid handler 9402 and place it in a predetermined location on the deck. Once the operator has placed the primary master well container 6, the operator starts an isolation/purification program running on computer 9412. This program drives the operations of liquid handler 9402 causing it to dispense magnetic particles 133 into all the wells of the primary master well container 6 containing lysed samples. Computer 9412 signals syringe pump 9414 to dispense the particles using pipettes 9410 into the primary master well container 6 when container 6 is in position 9424, away from the magnetic field created by magnet 9418.

Once the particles have been added, computer 9412 then directs the pipettes 9410 to add a chaotropic salt such as guadinium isothiocyanate to each of the wells to bind the genomic nucleic acid to the magnetic particles at 135. Once the chaotropic salt has been added, computer 9412 then mixes the contents of the wells by signaling the pipettes 9410 to alternately aspirate and redispense the material in each of the wells. This aspiration/redispensing process is preferably repeated three or four times to mix the contents in each well.

Once the contents of the wells have been mixed, computer 9412 pauses for two minutes to permit the particles, binding reagent, and raw biological material in the wells to incubate at room temperature in position 9424. When the two minutes have passed, computer 9412 commands the conveyor 9420 to move tray 9206 from position 9424 to position 9422, directly above magnet 9418 at 137. In this position the magnet draws the magnetic particles in each of the wells downward to the bottom of the wells of the primary master well container 6. Computer 9412 keeps tray 9206 and the primary master well container 6 over the magnet and within the magnetic field for 2-6 minutes, or until substantially all the magnetic particles are drawn to the bottom of each well and form a small pellet.

The particles drawn to the bottom of each well have genomic nucleic acid attached to their outer surface—genomic nucleic acid that the particles hold until an elution solution is placed in each well to release the genomic nucleic acid from the particles. With the particles at the bottom of each well and the wells located within the magnetic field, computer 9412 directs the pipettes to aspirate the supernatant 139.

Once the supernatant is removed, computer 9412 signals the conveyor to move the primary master well container 6 on tray 9206 to the nonmagnetic position 9424. The foregoing process of adding chaotropic salt, mixing the combination, pausing, drawing the magnetic particles down and aspirating the supernatant is repeated two more times.

Computer 9412 then directs the pipettes to introduce a wash solution (for example 70% ethanol when finctionalized beads are used, or 95% ethanol (4×) when silica beads are used) to resuspend the particles 141. Computer 9412 again mixes the contents of the wells by signaling the pipettes to alternately aspirate and redispense the material in each of the wells. With the wash buffer and particles thoroughly mixed, computer 9412 again moves tray 9206 and the primary master well container 6 back over magnet 9420 in position 9422 143 and draws the magnetic particles back to the bottom of the wells. This wash process 141, 143, 145 is repeated three times to thoroughly cleanse the magnetic particles, and dilute and remove all supernatant.

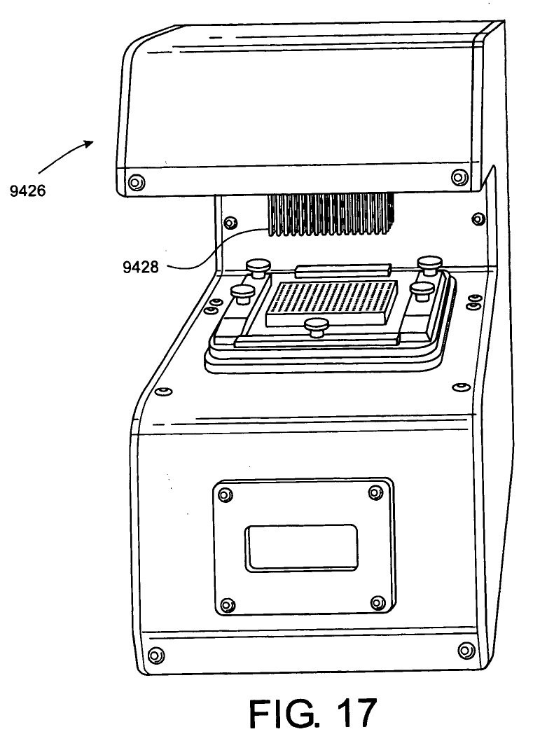

Once the particles are thoroughly washed, computer 9412 permits the magnetic particles in each well to air dry 147. In the preferred embodiment, shown in FIG. 17, the operator moves the primary master well container 6 to a dryer 9426 (an “Ultravap” dryer by Porvair Sciences, UK) having 384 tubules disposed in a 16×24 array 9428 that are configured to be simultaneously inserted into each of the wells of the primary master well container 6 and to supply warm, dry air thereto. In an alternative method, computer 9412 causes material handler 9408 to direct compressed dry nitrogen gas into each well of the primary master well container 6, drying the particles out in place while the container is in the magnetic field. Alternatively the samples can be permitted to air dry. Once the particles are completely dry, the primary master well container 6 can be subsequently moved away from the field of magnet 149.

Once the particles are dry, the operator returns the primary master well container 6 to the liquid handler 9402 and directs the computer 9412 to command the pipettes 9410 to fill the wells with an elution solution 151 and resuspend the particles. This elution solution is formulated to elute the bound genomic nucleic acid from the particles. An example of one such elution solution is 0.01M Tris (pH 7.4), sodium saline citrate (SSC), dimethyl sulfoxide (DMSO), sucrose (20%), 1×TBE, or formamide (100%). In the preferred embodiment, the elution solution is nuclease-free water. Nuclease free water is selected to minimize contamination and produce a standard concentration of purified genomic nucleic acid. In the preferred embodiment, the elution solution temperature is 22° C. A preferred yield is about 20 ng/μL of genomic nucleic acid is obtained.

After resuspending the genomic nucleic acid in a solution for a predetermined period of time, computer 9412 again moves tray 9206 with the primary master well container 6 via conveyor 9420 to position 9422 over magnet 9418 155. The magnet, in turn, draws the magnetic particles down to the bottom of each well. This leaves the genomic nucleic acid mixed and suspended in the elution solution. Computer 9412 then directs the pipettes to aspirate a small amount (50 μl) of purified genomic nucleic acid and to transfer 159 the small amount from each well into a corresponding well of a clean optical 384-well container that is also mounted on deck 9406. The operator scans 161 a barcode accession number on the optical container and computer 9412 transfers the scanned accession number to process controller 26, which then transfers it to LIMS 24. The operator takes this optical container to a UV spectrometer (Genios, by Tecan of Raleigh-Durham, N.C.), and directs the UV spectrometer to optically scan the optical container, by making an A₂₆₀ measurement 163. This measurement is electronically transferred 112 to LIMS 24 over a data communications link.

If another fully automated system is desired, the magnetic separator can be automated and rise from the bottom of the workstation and make contact with bottoms of all primary well containers simultaneously.

In the preferred embodiment for the biological sample, the genomic nucleic acid is not sonicated after separation from the cellular debris. The genomic nucleic acid includes at least a portion of intact nucleic acid. Unsonicated nucleic acid is recovered in the condition it is found in the lysate. Thus, if the genomic nucleic acid is intact in the lysate, it is intact (i.e., unfragmented) as attached to the particles. The sample contains at least a portion of intact genomic nucleic acid.

In certain types of samples, such as embryos, the genomic nucleic acid is substantially intact. In one embodiment, the genomic nucleic acid can be sonicated before or after separation with the magnetic particles. When the biological tissue is embryonic tissue sonication is preferred. Sonication can be done by any conventional means such as a fixed horn instrument or plate sonicator. In the one embodiment, the genomic nucleic acid is sonicated for five seconds to produce nucleic acid fragments. Although there is a wide range of fragments from about 100 base pairs to up to 20 kilobases, the average size of the fragment is around about 500 base pairs.

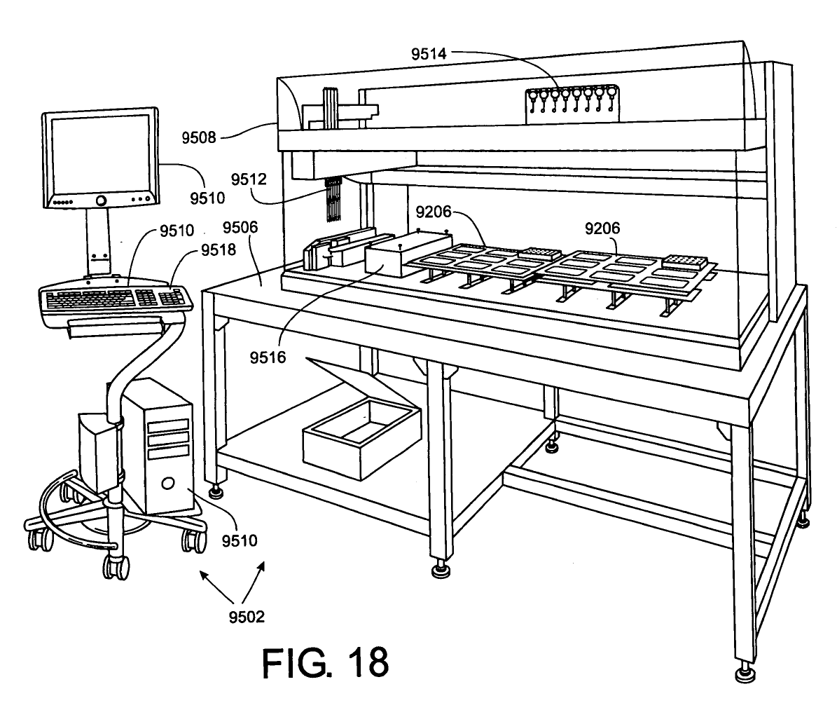

The primary master well container 6 is transported to the deck of the Screening Station 95 (FIG. 18) where its bar code is scanned 173. The operator places the container on a magnet, drawing all the magnetic particles to the bottom of the wells. The supernatant contains the purified genomic nucleic acid. LIMS 24 generates a worklist containing barcodes that list the primer combinations that need to be loaded onto the deck of the machine. The primer combinations are contained in barcoded tubes. An operator loads the barcoded tubes randomly into a primer box. The operator then scans the barcodes on the tubes using a Matrix scanner coupled to LIMS 24. The primer set combinations in the tubes are then loaded into an ABI 384 PCR plate (Applied Biosystems, Forest City, Calif.). The genomic nucleic acid sample from each well of the primary master well container 6 is added to a corresponding well of the ABI PCR plate that contains the primer combination or combinations appropriate to discern the relevant genotype 187. The ABI plate is then sealed with sealing tape and taken to the Detection Station 96 and placed in an ABI 7900. In the preferred embodiment the ABI 7900 cycles the ABI PCR plate 40 times between temperatures specified by the manufacturer. The operator can vary the number of cycles and the temperatures as desired to increase the signal provided by the samples.

FIG. 18 shows a preferred device for performing the Screening Station 95 functions. It comprises a liquid handler 9502 such as Genesis Tecan (Raleigh Durham, N.C.) or Multimeck Beckman (Indianapolis, Ind.). It includes a frame 9504, on which a deck 9506 is mounted to provide a horizontal working surface for first tray 9206 and second tray 9206. The first and second trays (as described above) can support and position nine primary master well containers 6.

Liquid handler 9502 also includes a material handler 9508 that is fixed to frame 9504 and extends upward and across the top surface of deck 9506. A computer 9510 is coupled to material handler 9508 to direct the movement and operation of pipettes 9512. Pipettes 9512 are fluidly coupled to a syringe pump 9514.

Primer block 9516 is disposed on the surface of deck 9506 and contains several tubes (not shown) each tube containing one or more combined primer sets. The operator bar-codes each tube and enters the data indicative of the tube contents (the particular primer in each tube, its volume and concentration) into LIMS 24, which stores the data associated with the bar code on the tube for later reference 173.

The operator places the primary master well containers 6 on deck 9506, scans the bar code accession number of the primary master well container 6, and signals computer 9510 to start transferring genomic nucleic acid and primer sets.

Based upon the information provided by the remote user 1, including the samples, the strains/profile for which the samples are to be tested, and the designated genetic sequences indicated by the strains/profile identification, as well as the primer sets necessary to detect those designated genetic sequences, as well as the location of each sample in the ABI PCR plate, LIMS 24 calculates a worklist that identifies for the operator which (and how many) tubes containing which primer sets must be placed in the primer block 9516 to test the samples in the primary master well container 6.

The operator first prints out this worklist, using it as a guide to identify and select particular tubes containing the proper primers. The operator takes these tubes out of storage, places them in the primer block 9516 and places the primer block 9516 on the Matrix scanner.

The Matrix scanner is coupled to LIMS 24, and is configured to scan the bar codes on each tube through holes in the bottom of the primer block. The scanner passes this information to LIMS, to which it is coupled, which in turn compares the bar codes of the scanned tubes with the bar codes of the primers identified on the worklist. Only if the operator has loaded the primer block with the appropriate type and number of primer sets will LIMS 24 permit the operator to proceed. In this manner, LIMS is configured to verify that the operator has inserted the appropriate and necessary tubes of primer sets into the primer block.

Once LIMS 24 has verified that the proper tubes of primer sets have been inserted into the primer block, it is configured to indicate to the operator that the primer block is acceptable and that the process steps at Screening Station 95 can begin.

The steps of preparing tubes of primer sets, entering them into LIMS, preparing a worklist, filling a primer block and verifying the primer block, all happen prior to the time the operator takes the primary master well container 6 with its 384 wells to the deck 9506 of liquid handler 9502 and places it in position on deck 9506.

The operator places the primary master well container 6 in position on first tray 9206 located on deck 9506 of liquid handler 9502. The operator electronically scans the container with an electronic scanner 9518 coupled to computer 9510 which, in turn, is coupled to process controller 26. As described above, the scanner may be any of several types of electronic scanner but is preferably a bar code scanner.

If there are several primary master well containers 6, they are preferably carried from the liquid handler of the Isolation/Purification Station 94 to the liquid handler of the Screening Station 95 in tray 9206, which can accommodate nine separate primary master well containers 6.

The operator also places a secondary master well container 27 (preferably an ABI 384 PCR plate) in a predetermined location on the second tray 9206 located on deck 9506 adjacent to the first tray 9206. The operator electronically scans the secondary master well container 27 with the electronic scanner 9518 and stores the location and identity of the secondary master well container 27 in process controller 26 which transmits the data to LIMS 24.

If there are several primary master well containers 6 that must be transferred to secondary master well containers 27, the corresponding secondary master well containers 27 may also be taken to liquid handler 9502 in trays 9206, rather than the operator carrying each secondary master well container 27 to second tray 9206 individually.

Once the operator places at least one primary master well container 6 in first tray 9506 and at least one secondary master well container 27 in second tray 9506, the operator signals computer 9510 to begin combining the primer sets and genomic nucleic acid extracted from the samples.

Generally speaking, computer 9510 commands material handler 9508 to extract primer sets from tubes in primer box 9516 and deposit them in each secondary master well container 27 in second tray 9206. Computer 9510 then commands material handler 9508 to extract the genomic nucleic acid from the wells of each primary master well container 6 in first tray 9206 and deposit the samples in wells in a corresponding secondary master well container 27. When the pipettes 9512 deposit the genomic nucleic acid samples and the primer sets in wells in the secondary master well containers 27, computer 9510 commands material handler 9508 and pipettes 9512 to mix the samples using the aspiration/redispensing methods discussed above.

The secondary master well containers 27 receive a number of aliquots of biological sample in multiple wells of the secondary master well container. In one embodiment, an aliquot of the biological sample of the strain is dispensed into at least four wells of the secondary master well container 27. To at least two of the four wells at least one primer set corresponding to at least one designated genetic sequence is added. A primer set correspond to a reference sequence is added to the third and fourth well. Thus, for example, if the genotype screening includes four designated genetic sequences, then four wells of the secondary master well containers 27 receive an aliquot of the biological sample and the corresponding primer sets for each designated genetic sequence. Additionally, four wells receive an aliquot of the biological sample and the corresponding four primer sets. This second set of wells is referred to as the replicants. The function of the replicants is quality control.

In a simpler embodiment, the validity of the screening data can be evaluated by dispensing an aliquot of a biological sample of the strain designated by the remote user into at least two wells of a microwell container. In one well at least one primer set is added corresponding to the at least one designated genetic sequence and to the other well at least one primer set is added corresponding to the reference sequence (SEQ ID NO. 1)

In an alternative embodiment an aliquot of biological sample of the strain designated by the remote user is dispensed into at least one well of a microwell container. In the one well multiple primer set with different fluorescently labeled primer sets are multiplexed together.

Furthermore, the detection of SNPs involves adding a primer set and two Real-Time PCR probes to a well.

Between one and five microliters of nucleic acid and four and fifteen microliters of primer sets are preferred to insure proper mixing of the samples and proper polymerization in the PCR process of the Detection Station 96 that follows.

Once the wells in the secondary master well containers 27 are filled with the appropriate purified genomic nucleic acid samples, primer sets, and these materials are mixed, computer 9510 signals the operator that the screening process is complete. The plate is then sealed with optical sealing tape. The operator then moves the secondary master well containers 27 to Detection Station 96 for further processing.

In the preferred embodiment, the central component of Detection Station 96 is the ABI 7900. The secondary master well containers 27 are placed inside the ABI 7900, where they are thermocycled 189 between 25 and 40 times. More particularly, the Detection Station 96 scans the secondary master well container's 27 barcode and reports it 196 to LIMS 24.

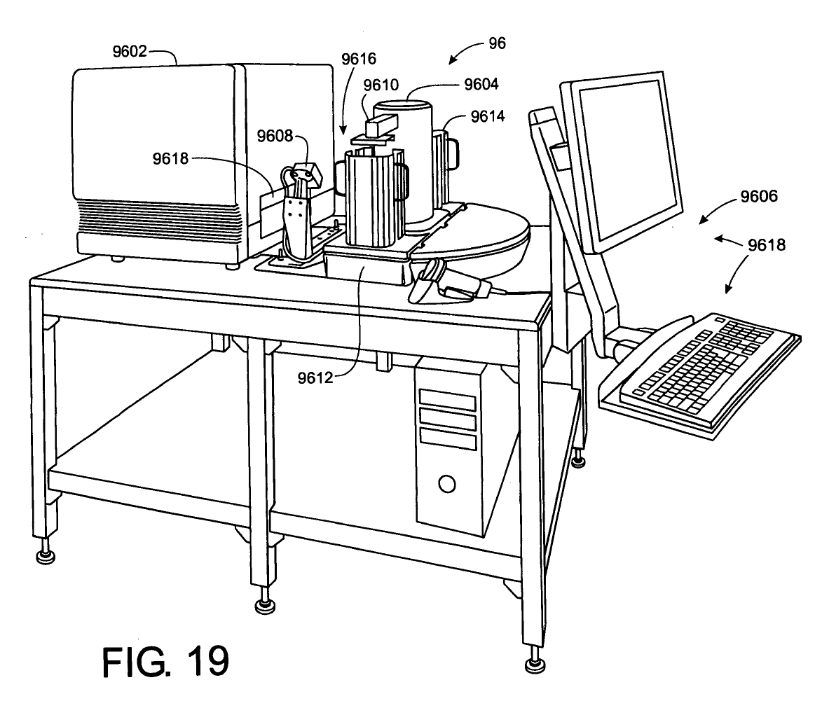

FIG. 19 illustrates a preferred device for performing the functions of Detection Station 96. It includes a PCR instrument 9602 (here shown as an ABI 7900), a material handler 9604 (here shown as a ZY mark arm), a computer 9606, and an electronic scanner 9608 (here shown as a barcode scanner).

Computer 9606 is coupled to PCR instrument 9602, material handler 9604, and process controller 26. It communicates with PCR instrument 9602 to control the insertion and removal of secondary master well containers 27 from PCR 9602 by handler 9604.

Scanner 9608 is coupled to handler 9604 to scan the accession numbers on the secondary master well containers 27, and to transmit those accession numbers to LIMS 24.

Material handler 9604 includes an arm 9610 that is commanded by computer 9606 to move between three positions: an incoming material hopper 9612, and outgoing material hopper 9614, and loading/unloading position 9616. Handler 9604 moves between these positions under the control of computer 9606, which commands this movement.

The operator first loads incoming material hopper 9612 with one or more secondary master well containers 27. The operator then operates the computer terminal 9618 of computer 9606, commanding computer 9606 to load and test the secondary master well containers 27. In response, computer 9606 commands arm 9610 to move to the incoming material hopper 9612, grasp the topmost secondary master well container 27, and to carry that container to the loading/unloading position 9616. Computer 9606 also commands PCR instrument 9602 to extend a tray (not shown) from an opening 9618 in the side of the ABI 7900, and commands arm 9610 to place the secondary master well container 27 on that tray. Scanner 9608 is configured to scan the barcode accession number on the secondary master well container 27, thereby making an electronic record of the secondary master well container 27 that is being tested. Scanner 9608 transmits this accession number to computer 9606, which later correlates the accession number with the test results provided by ABI 7900.

Once the secondary master well container 27 is placed in the tray, computer 9606 commands PCR instrument 9602 to retract the tray, and to begin processing the material in the secondary master well container 27, which is now inside PCR instrument 9602. PCR instrument 9602 signals computer 9606 when processing is complete. PCR instrument 9602 also transmits the processing results to computer 9606. Computer 9606, in turn, commands PCR instrument 9602 to eject the secondary master well container 27 that has just been processed, moving it back to loading/unloading position 9616. Once the secondary master well container 27 is in this position, computer 9606 commands material handler 9604 to move arm 9610 back to the loading/unloading position 9616 and to retrieve the secondary master well container 27 that has just been processed. Computer 9606 commands arm 9610 to move the just-processed secondary master well container 27 to outgoing material hopper 9614, where it is deposited, awaiting later removal by the operator of Detection Station 96.

The 384 PCR wellplate with the amplified DNA is moved to the deck of a Tecan Freedom Workstation. The deionized formamide/GeneScan-500[ROX] internal Lane size standard (ABI, #401734) solution and the AmpFLSTR® Profiler Plus® allelic ladder are also loaded onto the deck of the Tecan Workstation. The Tecan Genesis added the 1.5 μl amplified PCR products to the 25 μl of AmpFLSTR® reagents in a 384 Well PCR Plate. Other well locations in the 384 Well PCR Plate were loaded with 1.5 μl AmpFLSTR® Profiler Plus® allelic ladder to and 25 μl of the AmpFLSTR® reagents.

The 384 plate is then placed into a sample tray and placed on the autosampler of the capillary electrophoresis machine. The ABI prism 3100 Genetic Analyzer performs the auto loading, capillary electrophoresis and data capture of the samples.

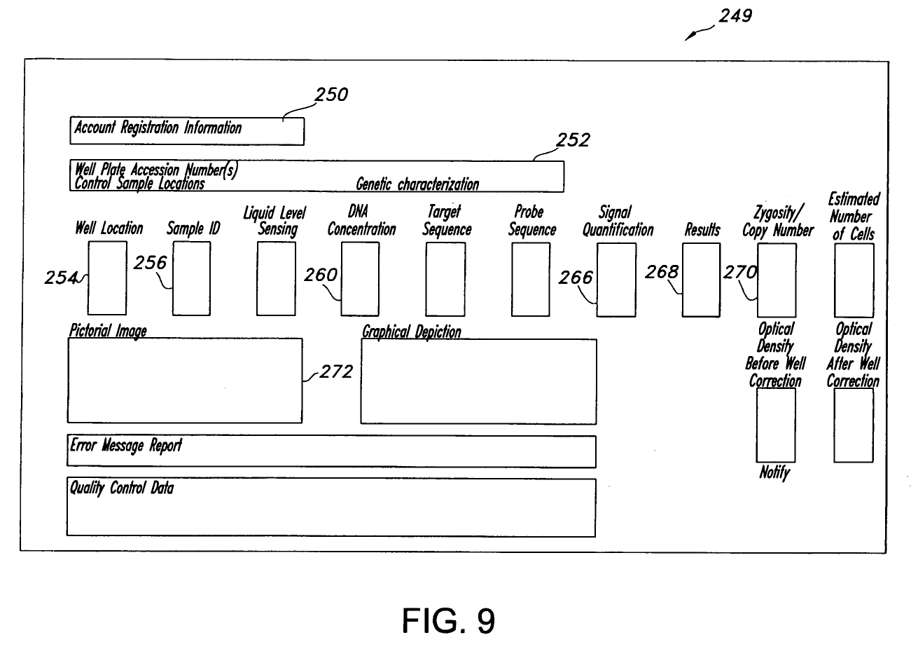

Now referring to FIG. 9, LIMS 24 now prepares the outcome report 249. Several calculations are performed before they are posted to the outcome report 249. In the preferred embodiment, such calculations include the evaluation of all replicates per sample, if replicated are performed. Calculating the relationship between the experimental quantified signal/values and the quantified signals/values of size standards.

A reference size standard is part of every run in order to normalize the data from every run. The resulting size standard values allows for the precise determination of fragment sizes of the designated genetic sequence being evaluated.

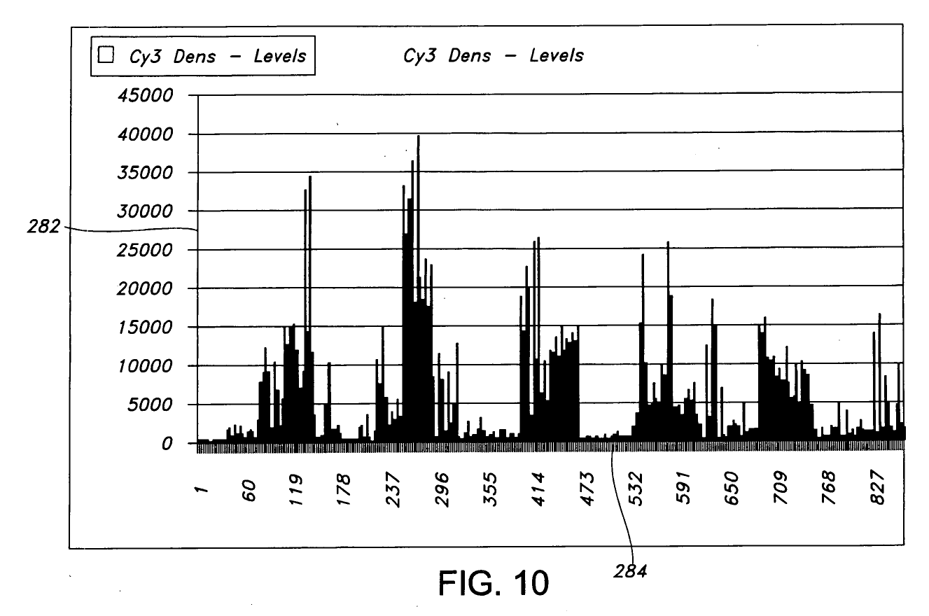

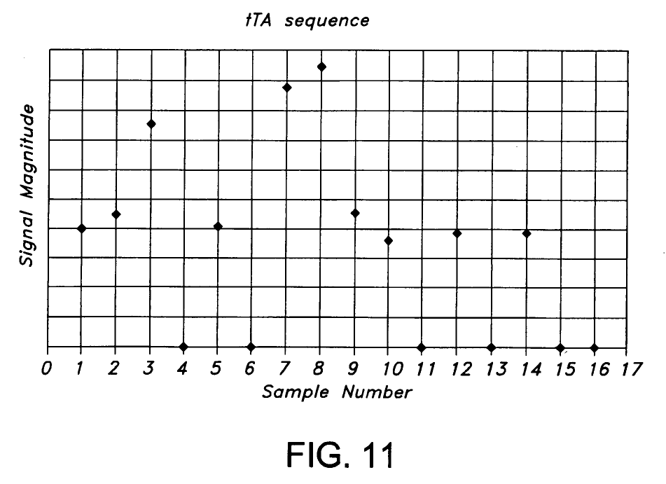

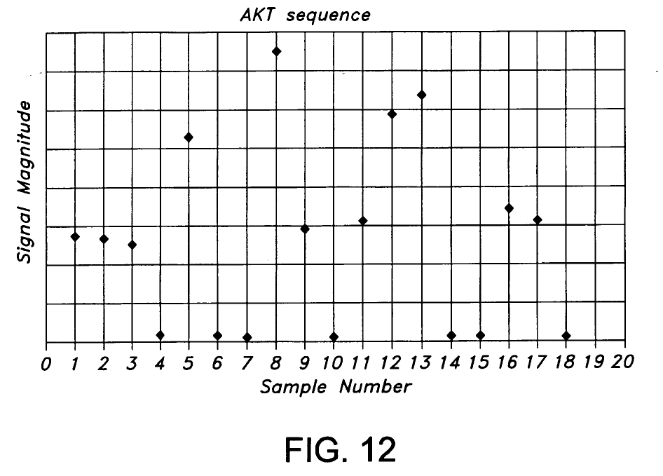

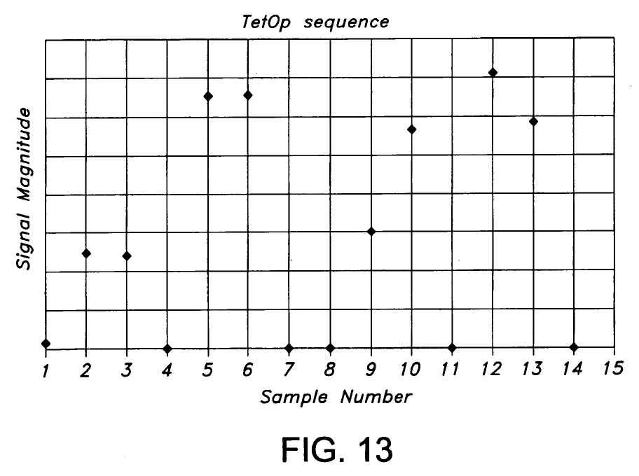

Now referring to FIG. 9, the sample outcome report 249 may include account registration 250, well plate container 2 barcode number(s) (i.e. accession numbers) 252, control sample locations 252 and genetic characterization of the designated control 252. Additionally, the outcome report 249 may include well location 254, sample identification 256, nucleic acid concentration 260, signal quantification 266, qualitative results 268, zygosity/copy number 270, and fragment sizes. The outcome report 249 may also include a picture file (email) or pictorial representations of results 272 as shown in FIG. 10. Additionally, information gathered at the request of the remote user 1 from optimization and sequence confirmation quality control data and error messages may be included in the outcome report 249. The remote user 1 may choose to have this file electronically sent or choose to be electronically notified. Additionally, remote user 1 has the option to have a hard copy sent via the postal service or facsimile.

Once the LIMS 24 has compiled all the data for the outcome report 249, the outcome report will be sent 7 to the remote user 1. In the preferred embodiment, LIMS 24 will send the report via a remote link 7 to either the remote user 1 or the order manager 22, which can post the results on the web site 16 or via an electronic link 7. The LIMS 24 will keep results available for six months and then the results will be recorded onto a long-term storage disk and archived.

The following examples are provided by way of examples and are not intended to limit the scope of the invention.

### EXAMPLES

### Example 1

Human Swab Sample Collection Method

The remote user 1 provides the genetic profile/line identification 84. The line includes at least one designated genetic sequence. The genetic line identification 84 has been previously associated with the designated genetic sequence that includes microsatellites such as D3S1358, v WA, FGA, and D8S1179, D21S11, D18S51, D5S818, D13S317, and D7S820. These microsatellites are included in the AmpFLSTR® Profiler Plus® kit (Applied Biosystems, Foster City, Calif.).

Microbrush® (Graftin, Wis.) Nylon Swabs, with biomatter adhered thereto are used to collect DNA samples from the oral cavities of humans. The swabs tips are removed and placed in individual wells of a VWR-DYNBL deep 96 well plate.

The four biological samples in the form of a frozen swabs is submitted via FedEx (Memphis, Tenn.) overnight delivery to the screening laboratory 20 from the remote user 1. Each sample occupies one well of a 96-well source well container. The biological samples are collected with swab 301 and swab holder 300.

A lysis reagent such Nuclei Lysing Solution (Promega Corporation, Madison, Wis. A7943) per sample) is gently poured into a 25 ml trough or reservoir and is placed on the deck of a Tecan Genesis Workstation (Research Triangle Park, N.C.). The liquid handler dispenses 150 μl of the lysis reagent in to each sample well of the source well container 2. The well plate is resealed and placed on a vortex for 10 minutes. The well plate is then placed back on the deck of the Tecan Genesis Workstation (Research Triangle Park, N.C.). The liquid handler aspirates 50 μl of each sample and dispenses it in to a 384 well primary master well container (Fisher Scientific #NC9134044). Once all of the samples are transferred, the primary master well container is moved to the deck of the Isolation Station Purification Station 94.

One-hundred and twelve microliters of SV Lysis reagent (Promega Corporation, Madison Wis., # Z305X) a chaotropic salt are added to each sample. Next, 13 μl of magnetic particles (Promega Corporation, #A220X) are added and the well components are mixed. The well plate is then moved into a magnetic field where the magnetic particles are drawn to the bottom of each well. The supernatant is then aspirated and discarded. The well plate is moved out of the magnetic field and 95 μl of SV Lysis reagent is added to each well and mixed. The well plate is then moved into the magnetic field and the supernatant is drawn off and discarded. This washing process is repeated two additional times. Next, the samples are washed four times in 130 μl of 95% ethanol as described above. After the fourth ethanol wash, the microwell container are placed on a 384 tip dryer for 11 minutes. Then the microwell container are moved back to the deck of the Isolation Station Purification Station 94 and 155 μl of Ambion's (Houston, Tex.) nuclease free water (catalog #B9934) is added to each well at room temperature. The plate is then moved into the magnetic field and 50 μl of DNA elution is transferred to a 384 well optical storage plate (Fisher Scientific, #08-772136) for optical density analysis. An A₂₆₀ reading of the storage plate read is performed with a Tecan Genios Spectrometer (Research Triangle Park, N.C.). This reading shows nucleic acid is present at the desired concentration of 0.2 O.D. units, but a range of 0.1 to 0.5 OD units is acceptable.

The primary master wellplate with the isolated DNA is moved to the deck of a Tecan Freedom Workstation. The AmpFLSTR® PCR Master Mix, AmpFLSTR® Profiler Plus® Primer Set and Taq DNA polymerase and Ambion water are placed on the deck as well. The final PCR mixture is made of 1×AmpFLSTR® PCR Master Mix, 1×AmpFLSTR®& Profiler Plus® Primer Set and 40% isolated DNA. The Tecan Genesis added the reagents together in the 384 Well PCR Plate. The plate is then sealed with optical sealing tape (ABI, #4311971).

The samples are then placed in an Applied Biosystems SDS 7900. A standard PCR protocol is followed by heating the samples to 95° C. for 11 minutes, followed by thermally cycling the samples 28 times between 94° C. for one minute, 59° C. for one minute and 72° C. for one minute. The thermal cycling is followed by a final extension step of 60° C. for 45 minutes. The final step is at 25° C. for an indefinite period of time.

The PCR wellplate with the amplified DNA is moved to the deck of a Tecan Freedom Workstation. The deionized formamide/GeneScan-500[ROX] internal Lane size standard (ABI, #401734) solution and the AmpFLSTR® Profiler Plus® allelic ladder are also loaded onto the deck of the Tecan Workstation. The Tecan Genesis added the 1.51 μl amplified PCR products to the 25 μl of AmpFLSTR® reagents in a 384 Well PCR Plate. Other well locations in the 384 Well PCR Plate were loaded with 1.5 μl AmpFLSTR® Profiler Plus® allelic ladder to and 25 μl of the AmpFLSTR® reagents.

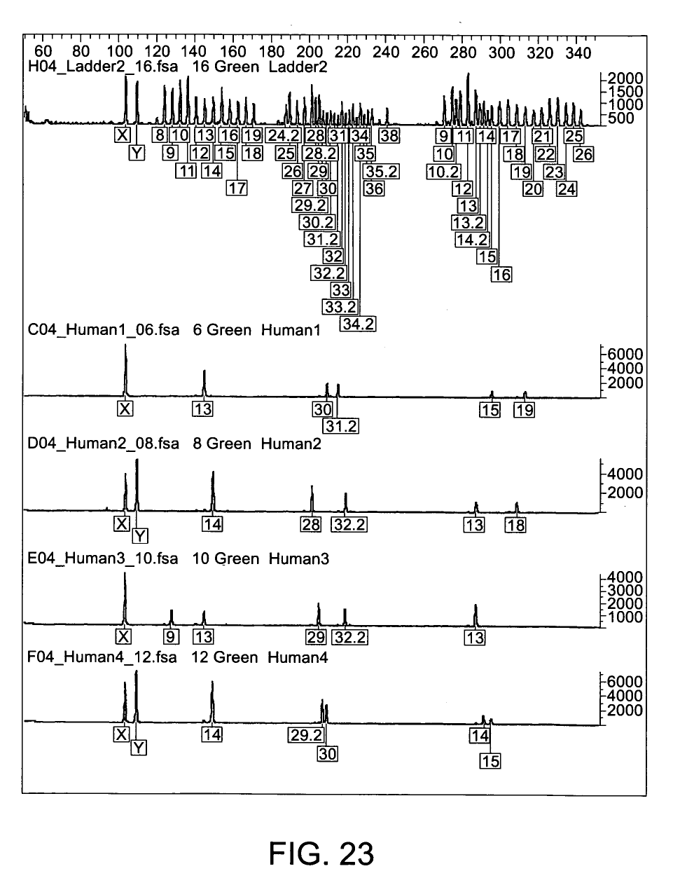

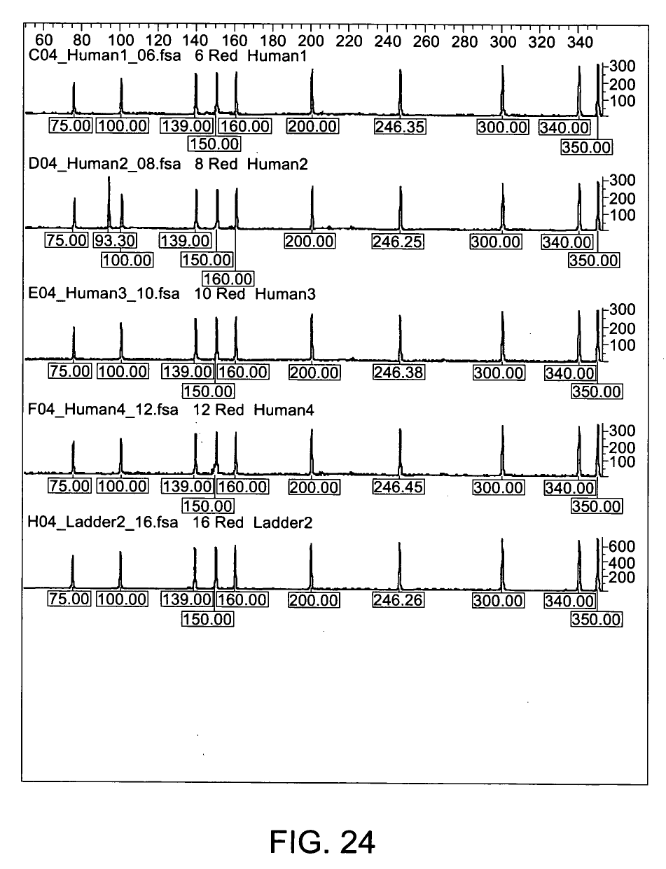

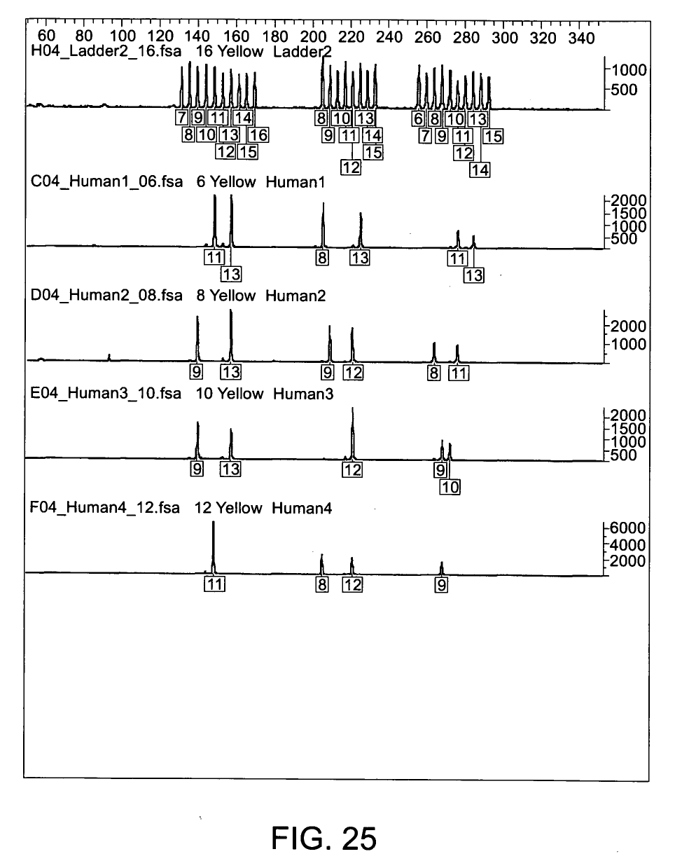

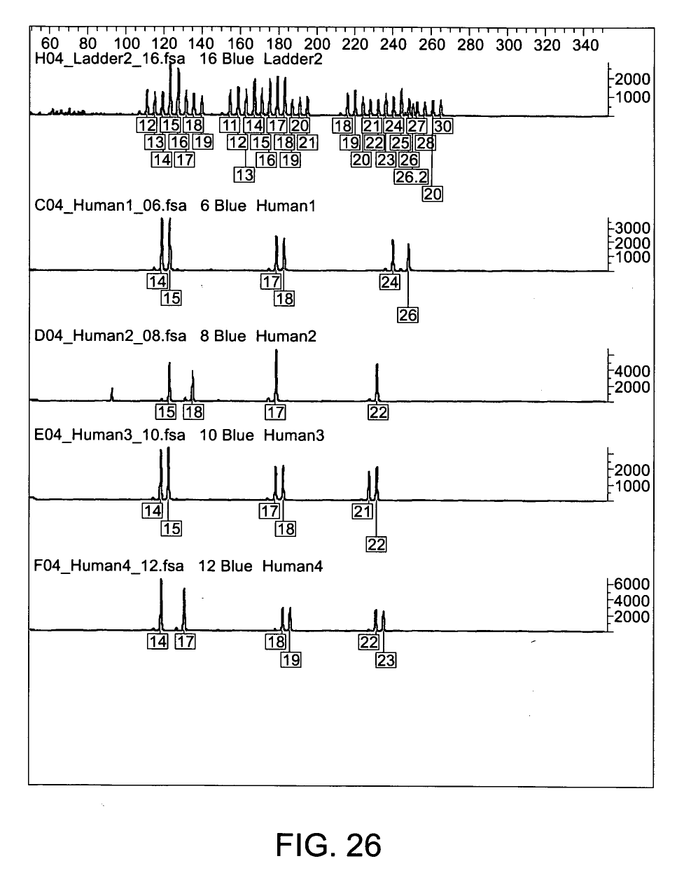

The 384 plate is then placed into a sample tray and placed on the autosampler of the capillary electrophoresis machine. The ABI prism 3100 Genetic Analyzer performs the auto loading, capillary electrophoresis and data capture of the samples. On average, these results are transmitted to the remote user 1 within twenty-four hours of receiving the biological sample at the screening laboratory 20. The results are shown in Table 2 and FIGS. 23-26.

TABLE 2

| | Human | Human | Human | Human |
|---|---|---|---|---|
| Locus (STR) | DNA 1 | DNA 2 | DNA 3 | DNA 4 |
| D3S1358 | 14, 15 | 15, 18 | 14, 15 | 14, 17 |
| vWA | 17, 18 | 17 | 17, 18 | 18, 19 |
| FGA | 24, 26 | 22 | 21, 22 | 22, 23 |
| D8S1179 | 13 | 14 | 9, 13 | 14 |
| D21S11 | 30, 31.2 | 28, 32.2 | 29, 32.2 | 29.2, 30 |
| D18S51 | 15, 19 | 13, 18 | 13 | 14, 15 |
| D5S818 | 11, 13 | 9, 13 | 9, 13 | 11 |
| D13S317 | 8, 13 | 9, 12 | 12 | 8, 12 |
| D7S820 | 11, 13 | 8, 11 | 9, 10 | 9 |
| AMELOGENIN | X, X | X, Y | X, X | X, Y |

### Example 2

Congenics Example

The remote user 1 provides the genetic profile identification 84. A profile includes at least one designated genetic sequence. The genetic profile identification name 84 has been previously associated with the designated genetic sequence that includes microsatellites such as D1Mit495, D2Mit208, D2Mit155, D2Mit1, D3Mit51, and D4Mit203.

A biological sample in the form of a mouse tail tissue biopsy is submitted via FedEx (Memphis, Tenn.) overnight delivery to the screening laboratory 20 from the remote user 1. Each sample occupies one well of a 96-well source well container.

A lysis reagent (made of 2.5 μl of proteinase K (VWR EM-24568-3) and 147.5 μl of Nuclei Lysing Solution (Promega Corporation, Madison, Wis. A7943) per sample) is gently mixed and poured into a 25 ml trough or reservoir and is placed on the deck of a Tecan Genesis Workstation (Research Triangle Park, N.C.). The liquid handler dispenses 150 μl of the lysis reagent in to each sample well of the source well container 2. The well plate is then placed in a 55° C. oven for three hours. The well plate is then placed back on the deck of the Tecan Genesis Workstation (Research Triangle Park, N.C.). The liquid handler aspirates 50 μl of each sample and dispenses it in to a 384 well primary master well container (Fisher Scientific #NC9134044). Once all of the samples are transferred, the primary master well container is moved to the deck of the Isolation Station Purification Station 94.

One-hundred and twelve microliters of SV Lysis reagent (Promega Corporation, Madison Wis., # Z305X), a chaotropic salt, are added to each sample. Next, 13 μl of magnetic particles (Promega Corporation, #A220X) are added and the well components are mixed. The well plate is then moved into a magnetic field where the magnetic particles are drawn to the bottom of each well. The supernatant is then aspirated and discarded. The well plate is moved out of the magnetic field and 95 μl of SV Lysis reagent is added to each well and mixed. The well plate is then moved into the magnetic field and the supernatant is drawn off and discarded. This washing process is repeated two additional times. Next, the samples are washed four times in 130 μl of 95% ethanol as described above. After the fourth ethanol wash, the microwell container is placed on a 384 tip dryer for 11 minutes. Then the microwell container is moved back to the deck of the Isolation Station Purification Station 94 and 155 μl of Ambion's (Houston, Tex.) nuclease free water (catalog #B9934) is added to each well at room temperature. The plate is then moved into the magnetic field and 50 μl of DNA elution is transferred to a 384 well optical storage plate (Fisher Scientific, #08-772136) for optical density analysis. An A₂₆₀ reading of the storage plate is performed with a Tecan Genios Spectrometer (Research Triangle Park, N.C.). This reading shows nucleic acid is present at the desired concentration of 0.2 O.D. units, but a range of 0.1 to 0.5 OD units is acceptable.

The primary master wellplate with the isolated DNA is moved to the deck of a Tecan Freedom Workstation. The Master Mix, PCR primer mixture and Ambion water are placed on the deck as well. The final PCR mixture is made of 1×Master Mix (catalog # 4326708), 1×PCR primer mix for a designated genetic sequence (Applied Biosystems, D1Mit495 #4322998, D2Mit208 #4323129, D2Mit155 #4323007, D2Mit1 #4323120, D3Mit51 #4323141, and D4Mit203 #4323225) and 8% isolated DNA. The Tecan Genesis adds the reagents together in the ABI 7900 384 Well Optical Plate. The plate is then sealed with optical sealing tape (ABI, #4311971).

The samples are then placed in an Applied Biosystems SDS 7900. A standard PCR protocol is followed by heating the samples to 50° C. for two minutes then incubated at 95° C. for 12 minutes, followed by thermally cycling the samples 10 times between 94° C. for 45 seconds, 55° C. for one minute and 72° C. for one minute. The samples were then thermocycled for 15 times between 89° C. for one minute, 55° C. for one minute and 72° C. for one minute. Following thermocycling the samples are held at 72° C. for ten minutes.

The PCR wellplate with the amplified DNA is moved to the deck of a Tecan Freedom Workstation. The deionized formamide/GeneScan-500[ROX] internal Lane size standard (ABI, #401734) solution and the AmpFLSTR® Profiler Plus® allelic ladder are also loaded onto the deck of the Tecan Workstation. The Tecan Genesis added the 1.5 μl amplified PCR products to the 25 μl of AmpFLSTR® reagents in a 384 Well PCR Plate. Other well locations in the 384 Well PCR Plate were loaded with 1.5 μl AmpFLSTR® Profiler Plus® allelic ladder to and 25 μl of the AmpFLSTR® reagents.

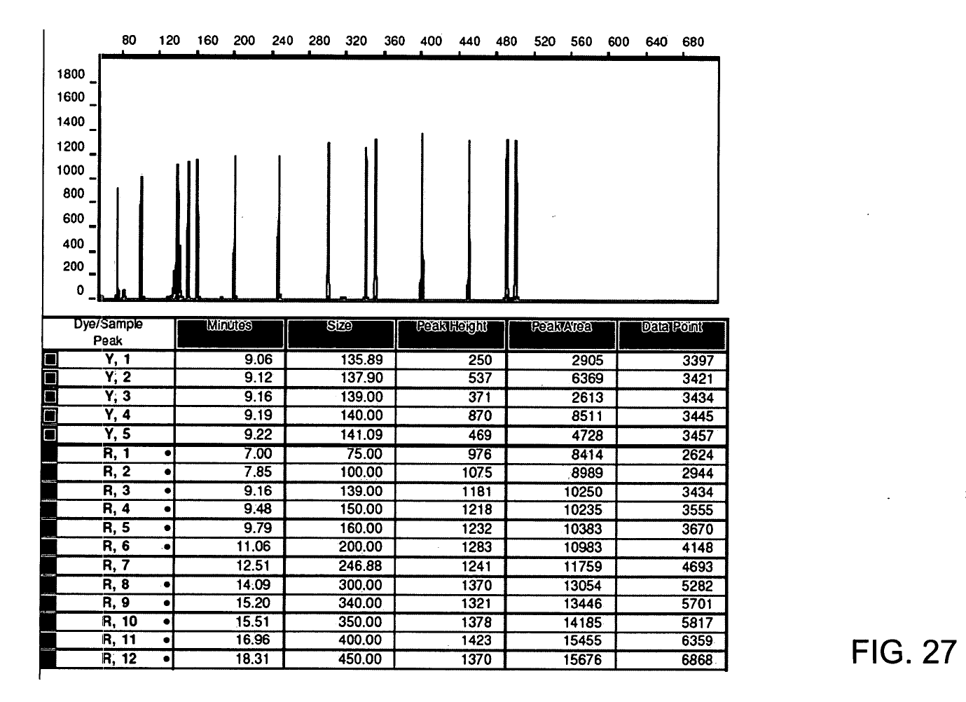

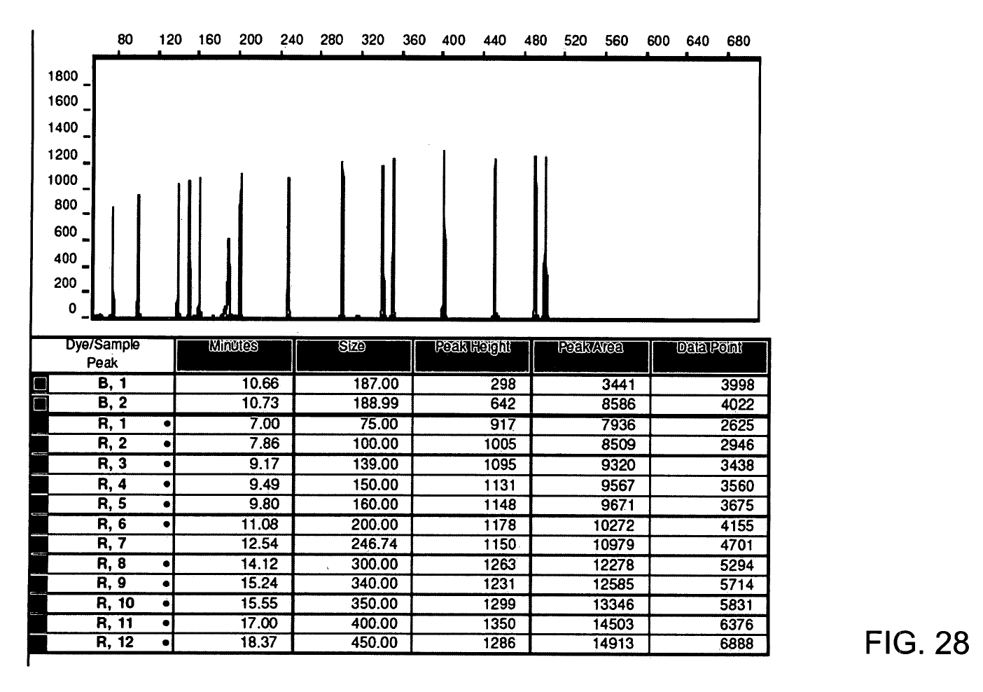

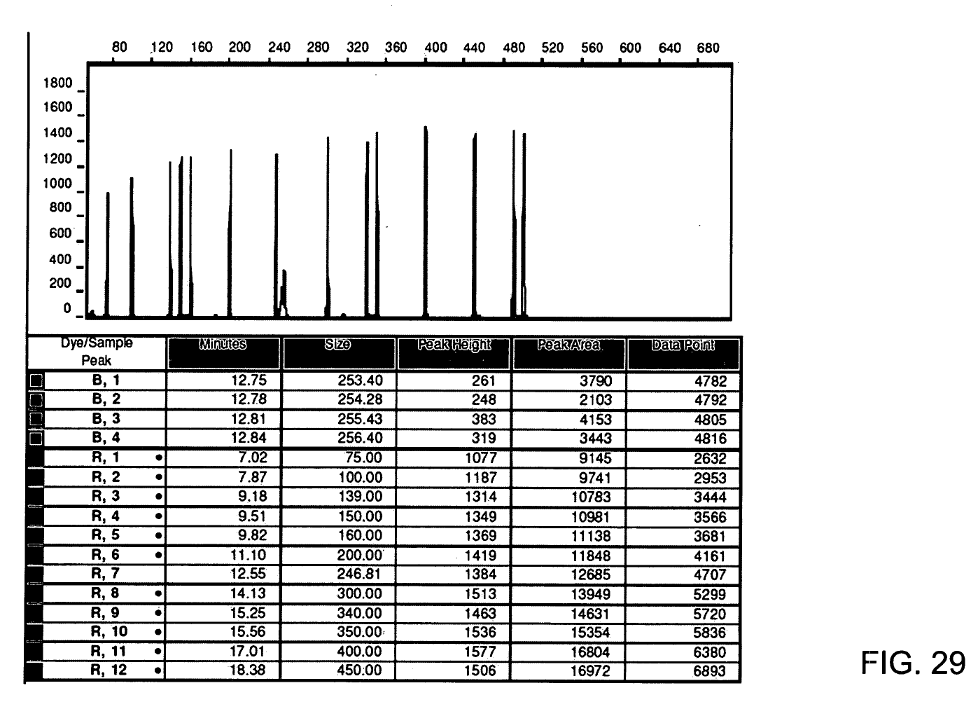

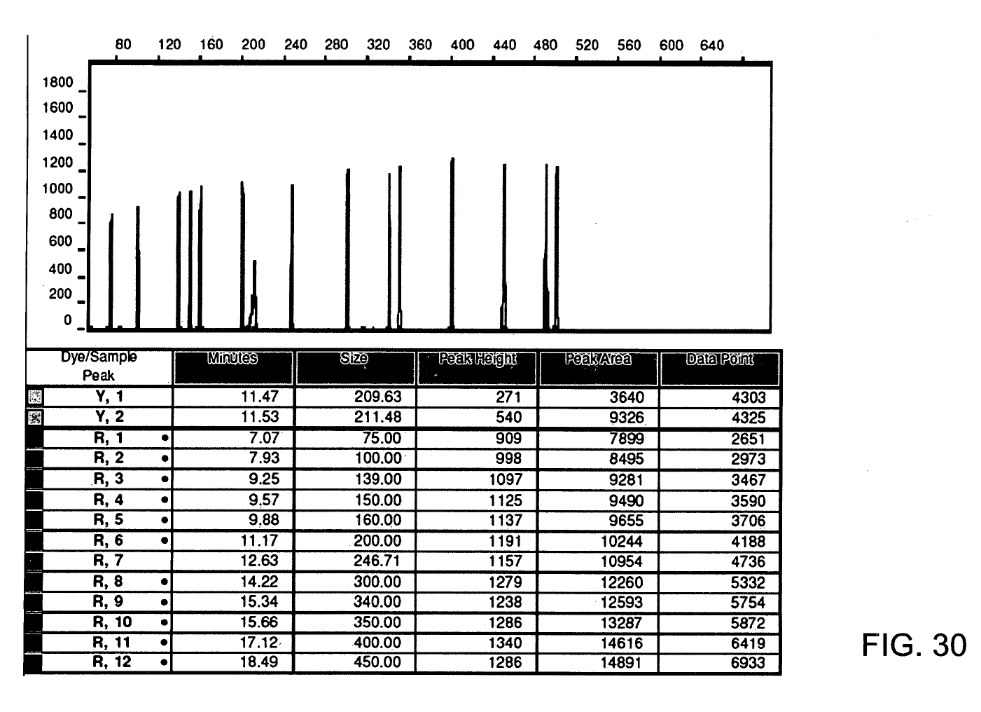

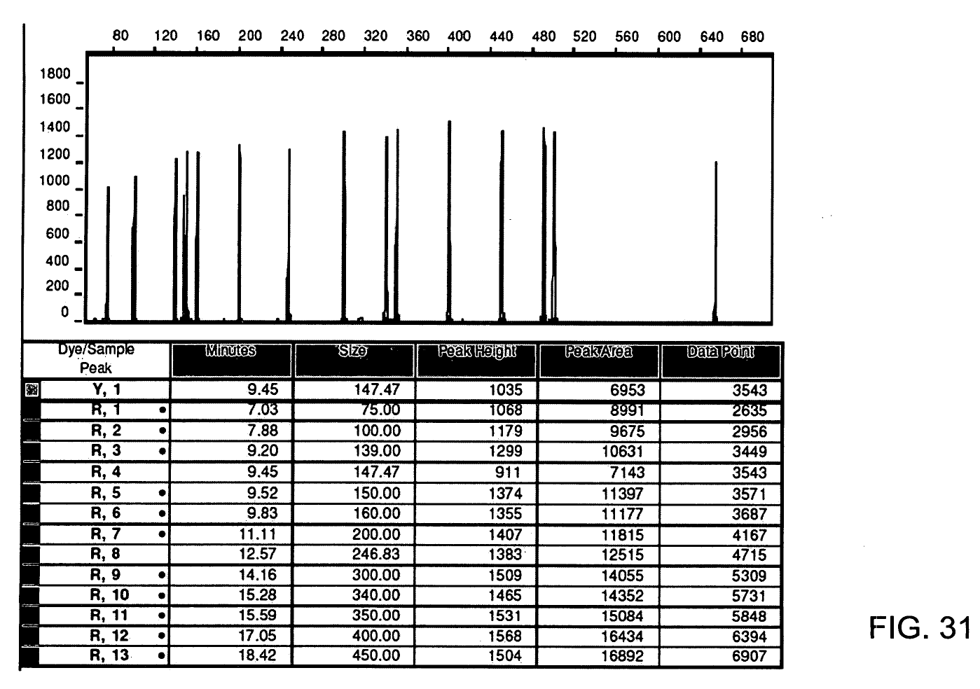

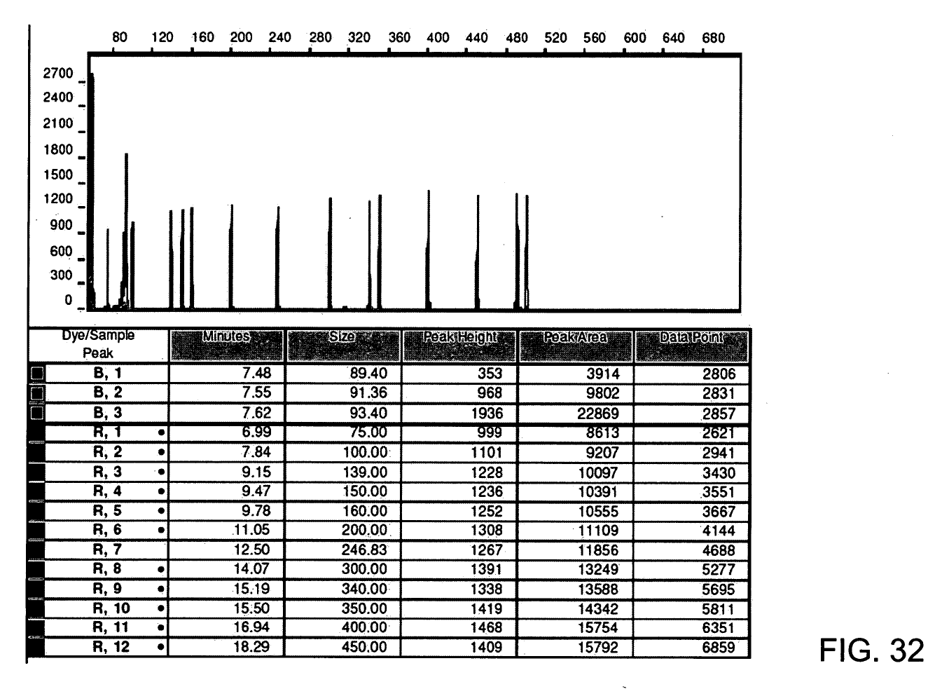

The 384 plate is then placed into a sample tray and placed on the autosampler of the capillary electrophoresis machine. The ABI prism 3100 Genetic Analyzer performs the auto loading, capillary electrophoresis and data capture of the samples. On average, these results are transmitted to the remote user 1 within twenty-four hours of receiving the biological sample at the screening laboratory 20. The screening results are shown in FIGS. 27-32.

### Example 3

Congenic Murine Blood Sample Collection Method

The remote user 1 provides the genetic profile/line identification 84. The line includes at least one designated genetic sequence. The genetic line identification 84 is associated with the designated genetic sequence that includes microsatellite D1Mit495.

Specifically, a remote user 1 can contact the screening laboratory 20 and provide a name of the microsatellite. The microsatellite name may be used to query databases to yield literature specific for this microsatellite by the screening laboratory 20. The Mouse Genome Informatics (MGI) databases with their respective hyper links, yield the following database reference: AC132109. Reports Mus musculus BAC . . . [gi:3314745] which yields the designated genetic sequence.

(SEQ ID NO. 5)

CCTTTGGTCTCTGGAGTGCTGGTGTATGCTGAAAGTGATCAAAAGAGTTT
CTCTTCCCCCCATGTCCCCATCCTTCAGAAGTGAAGGGGAGCAGCCCTGG
GCCCTGCTCTGGCGGATGCTGTGGTGGGAAGGAAGTGGCTAAAGGGTTGC
TGGGCCTCTTCTTTGAGACCTTAGTGGTGGTGTCTTTCATGTAGCACATG
CTGGAGGCTGGGAGCCAGTGAGTCTTTCCTGTGAGGGGTCTTTCAGGAGC
TGAATTGCTCAGCAGACCTGAATGAAGAAATGGCTACATGTAAGCCAGGT
CCACCTTGCTCCAAAAGAAAGTGATTCTCTCTCTCTCTCTCTCTCTCTCT
CACACACACACACACACACACACACACACACACAACTTTCTTTATAGTTT
TATTGTGGCAGCCTCTCAGAGGGTCAGTTGTTTGTTTAAGATACACTCAA
TTAATGATATGCTTGCTTCATATTGTGTCTTTCAGAGGAGAAAGGTAACT
GAGACCCCACTGCTCACCCGCTGTATTGTTAAGCCAGTGAATAGGAACCC
AAAAGGACAGAGAAGACACCCAACTTAACTCACCATGCACGAGTCTCTGA
GCCTCAGGAACTTGAATTTA

Upon identification of the designated genetic sequence two other software programs are utilized. The first of these programs is a blast program that identifies homologies between the designated genetic sequence and the endogenous genome of the mouse, as well as other species. The blast software can be found at http://www.ncbi.nlm.nih.gov/BLAST/.

The second of these programs is repeat masking program, such as Repeat Master Web Server found at http://www.repeatmasker.org/cgi-bin/WEBRepeatMasker. This program identifies areas in the designated genetic sequence that are highly repetitive, making them less than ideal locations to build a primer probe. If such areas are found in the designated genetic sequence they are masked by replacing the normal nucleotide designation A,C,G or T with the letter N or X.

A PCR primer design software program, such as Primer Express®, is used by the screening laboratory to identify primer sequences that will detect this genetic condition. The software generates the following primers.

| | |
|---|---|
| Forward Primer: CCACCTTGCTCCAAAAGAAA | (SEQ ID NO. 6) |
| Reverse Primer: TATTGTGGCAGCCTCTCAGA | (SEQ ID NO. 7) |

The primers and probes will hybridized or anneal the following areas in the designated genetic sequence.

(SEQ ID NO.5)

CCTTTGGTCTCTGGAGTGCTGGTGTATGCTGAAAGTGATCAAAAGAGTTT
CTCTTCCCCCCATGTCCCCATCCTTCAGAAGTGAAGGGGAGCAGCCCTGG
GCCCTGCTCTGGCGGATGCTGTGGTGGGAAGGAAGTGGCTAAAGGGTTGC
TGGGCCTCTTCTTTGAGACCTTAGTGGTGGTGTCTTTCATGTAGCACATG
CTGGAGGCTGGGAGCCAGTGAGTCTTTCCTGTGAGGGGTCTTTCAGGAGC
TGAATTGCTCAGCAGACCTGAATGAAGAAATGGCTACATGTAAGCCAGGT
CCACCTTGCTCCAAAAGAAAGTGATTCTCTCTCTCTCTCTCTCTCTCTCT
CACACACACACACACACACACACACACACACACAACTTTCTTTATAGTTT
TATTGTGGCAGCCTCTCAGAGGGTCAGTTGTTTGTTTAAGATACACTCAA
TTAATGATATGCTTGCTTCATATTGTGTCTTTCAGAGGAGAAAGGTAACT
GAGACCCCACTGCTCACCCGCTGTATTGTTAAGCCAGTGAATAGGAACCC
AAAAGGACAGAGAAGACACCCAACTTAACTCACCATGCACGAGTCTCTGA
GCCTCAGGAACTTGAATTTA

The genomic DNA nucleotides from the forward primer to the end of the reverse primer and all the bases in between, whether they hybridized to primer probe are not, are known as the target genetic sequence. For D1MIT495 the target genetic sequence is:

(SEQ ID NO. 5)

CCTTTGGTCTCTGGAGTGCTGGTGTATGCTGAAAGTGATCAAAAGAGTTT
CTCTTCCCCCCATGTCCCCATCCTTCAGAAGTGAAGGGGAGCAGCCCTGG
GCCCTGCTCTGGCGGATGCTGTGGTGGGAAGGAAGTGGCTAAAGGGTTGC
TGGGCCTCTTCTTTGAGACCTTAGTGGTGGTGTCTTTCATGTAGCACATG
CTGGAGGCTGGGAGCCAGTGAGTCTTTCCTGTGAGGGGTCTTTCAGGAGC
TGAATTGCTCAGCAGACCTGAATGAAGAAATGGCTACATGTAAGCCAGGT
CCACCTTGCTCCAAAAGAAAGTGATTCTCTCTCTCTCTCTCTCTCTCTCT
CACACACACACACACACACACACACACACACACAACTTTCTTTATAGTTT
TATTGTGGCAGCCTCTCAGAGGGTCAGTTGTTTGTTTAAGATACACTCAA
TTAATGATATGCTTGCTTCATATTGTGTCTTTCAGAGGAGAAAGGTAACT
GAGACCCCACTGCTCACCCGCTGTATTGTTAAGCCAGTGAATAGGAACCC
AAAAGGACAGAGAAGACACCCAACTTAACTCACCATGCACGAGTCTCTGA
GCCTCAGGAACTTGAATTTA

Mouse tails are nicked with a razor blade and the resulting blood droplets are blotted on to filter paper (V&P Scientific Lint Free Blotting Media (114 mm long, 74 mm wide) #VP540D). The samples are placed in individual wells of a Nunc 96-well plate (Fisher Scientific 12-565-368). The well locations are labeled and the plates are transported shipped to the screening laboratory 20.

The number of samples are counted and lysis reagent is made (2.5 μl of proteinase K (VWR EM-24568-3) and 147.5 μl of Nuclei Lysing Solution (Promega Corporation, Madison Wis., A7943) per sample. The solution is gently mixed and poured into a 25 ml trough or reservoir and placed on the deck of a Tecan Genesis Workstation (Research Triangle Park, N.C.). The liquid handler dispenses 150 μl of the solution into each sample well. The well plate is then placed in a 55° C. oven for three hours.

The well plate is then placed back on the deck of the Tecan Genesis Workstation. The liquid handler aspirates 50 μl of each sample and dispenses it in to a 384 primary master well container (Fisher Scientific #NC9134044). Once all of the samples are transferred, the primary master well container is moved to the deck of the Isolation Station Purification Station 94.

One-hundred and twelve microliters of SV Lysis reagent (Promega Corporation, #Z305X) are added to each sample. Next, 13 μl of magnetic particles (Promega Corporation #A220X) are added and the well components are mixed. The well plate is then moved into a magnetic field where the magnetic particles are drawn to the bottom of each well. The supernatant is then aspirated and discarded. The well plate is moved out of the magnetic field and 95 μl of SV Lysis reagent is added to each well and mixed. The well plate is then moved into the magnetic field and the supernatant is drawn off and discarded. This washing process is repeated two additional times. Next, the samples are washed four times in 130 μl of 95% ethanol as described above. After the last ethanol wash, the well plate is placed on a 384 tip dryer for 11 minutes. Then the well plate is moved back to the deck of the Isolation Station and 155 μl of Ambion's (Houston, Tex.) nuclease free water (catalog #B9934) is added to each well. The elution solution is heated to 95°. The plate is then moved into the magnetic field and 50 μl of DNA elution is transferred to a 384 well optical storage plate (Fisher Scientific, #08-772136) for optical density analysis.

An A₂₆₀ reading of the storage plate read is performed with a Tecan Genios Spectrometer. This reading shows nucleic acid is present at the desired concentration of 0.2 O.D. units, but, a range of 0.1 to 0.5 O.D. units is acceptable.

The primary master wellplate with the isolated DNA is moved to the deck of a Tecan Freedom Workstation. The AmpFLSTR® PCR Master Mix, AmpFLSTR® Profiler Plus® Primer Set and Taq DNA polymerase and Ambion water are placed on the deck as well. The final PCR mixture is made of 1×AmpFLSTR® PCR Master Mix, 1×AmpFLSTR® Profiler Plush® Primer Set and 40% isolated DNA. The Tecan Genesis added the reagents together in the 384 Well PCR Plate. The plate is then sealed with optical sealing tape (ABI, #4311971).

The samples are then placed in an Applied Biosystems SDS 7900. A standard PCR protocol is followed by heating the samples to 95° C. for 11 minutes, followed by thermally cycling the samples 28 times between 94° C. for one minute, 59° C. for one minute and 72° C. for one minute. The thermal cycling is followed by a final extension step of 60° C. for 45 minutes. The final step is at 25° C. for a period of time.

The PCR wellplate with the amplified DNA is moved to the deck of a Tecan Freedom Workstation. The deionized formamide/GeneScan-500[ROX] internal Lane size standard (ABI, #401734) solution and the AmpFLSTR® Profiler Plus® allelic ladder are also loaded onto the deck of the Tecan Workstation. The Tecan Genesis added the 1.5 μl amplified PCR products to the 25 μl of AmpFLSTR® reagents in a 384 Well PCR Plate. Other well locations in the 384 Well PCR Plate were loaded with 1.5 μl AmpFLSTR® Profiler Plus® allelic ladder to and 25 μl of the AmpFLSTR® reagents.

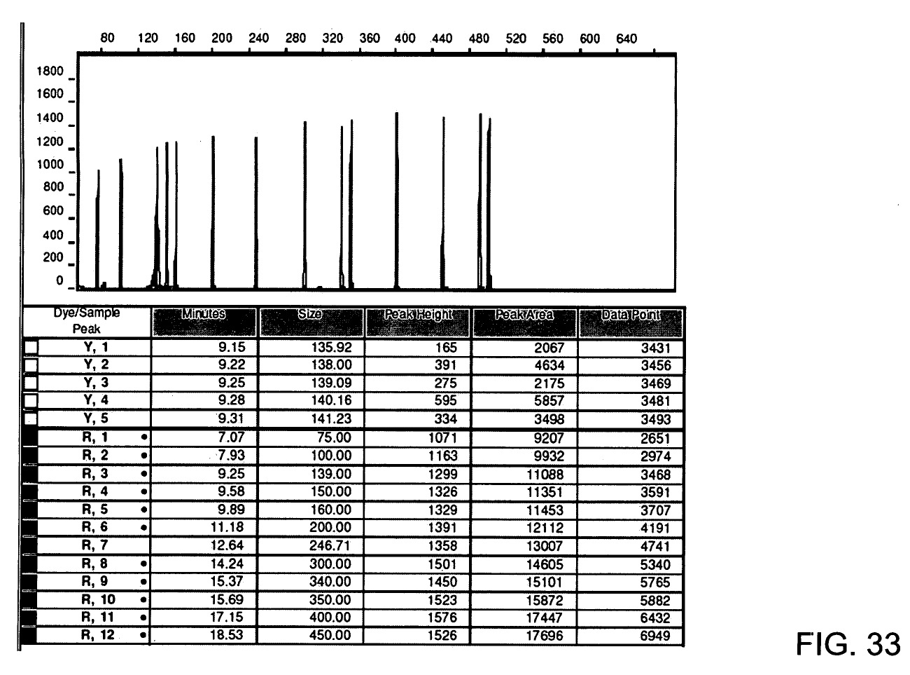

The 384 plate is then placed into a sample tray and placed on the autosampler of the capillary electrophoresis machine. The ABI prism 3100 Genetic Analyzer performs the auto loading, capillary electrophoresis and data capture of the samples. On average, these results are transmitted to the remote user 1 within twenty-four hours of receiving the biological sample at the screening laboratory 20. The results are shown in FIG. 33.

Although the present invention has been described and illustrated with respect to preferred embodiments and a preferred user thereof, it is not to be so limited since modifications and changes can be made therein which are within the full scope of the invention.

1. A method to screen a plurality of samples for a microsatellite loci comprising the steps of:
acquiring the identity of at least one microsatellite loci for a said plurality of samples;
obtaining means to determine the presence of said microsatellite loci;
receiving at a screening laboratory from a remote user a plurality of samples each disposed in a designated well of a microwell container, and screening said plurality of samples for at least one microsatellite loci.

2. The method of claim 1 further including the step of:
reporting a screening result to the remote user twenty-four hours of receiving said sample at said screening laboratory.

3. The method of claim 1 wherein said sample is murine.

4. The method of claim 1 wherein said sample is obtained from a human.

5. The method of claim 1 wherein said means to determine the presence of said microsatellite loci is a primer set.

6. The method of claim 1 wherein said plurality of samples are each disposed on an absorbent carrier and said absorbent carrier is disposed in a well of a microwell container.

7. The method of claim 1 wherein said screening result have forensic applications.

8. The method of claim 1 wherein said screening result have congenic application.
